SEQUENCE LISTING (9 sequences)

SEQ ID NO: 1 - RNA, 7 nt, Homo sapiens
acauuca

SEQ ID NO: 2 - RNA, 23 nt, Homo sapiens
aacauucaacgcugucggugagu

SEQ ID NO: 3 - DNA, 20 bp, Homo sapiens
accatcgaccgttgattgta

SEQ ID NO: 4 - DNA, 17 bp, Homo sapiens
ttggaattcaaataaaa

SEQ ID NO: 5 - DNA, 15 bp, Homo sapiens
ttgggatttgaaaaa

SEQ ID NO: 6 - DNA, 60 bp, Homo sapiens
aacattcaacgctgtcggtgagtttggaattcaaataaaaaccatcgaccgttgattgta

SEQ ID NO: 7 - DNA, 110 bp, Homo sapiens
agaagggctatcaggccagccttcagaggactccaaggaacattcaacgctgtcggtgagtttgggatttgaaaaaaccactgaccgttgactgtaccttggggtcctta

SEQ ID NO: 8 - DNA, 388 bp, Homo sapiens
ctcgagtgtgacaggtttggttaaaggattgggctttcctctgcctccctcctgctccagactcccacagatactgtttaaatcagcacatctctgcctcacaggttgcttcagtgaacattcaacgctgtcggtgagtttggaattcaaataaaaaccatcgaccgttgattgtaccctatagctaaccatcatctactccatggccctctgcgtttgctgaagacagaaccgcaaagcaggacccgacaggattcttttttaattaagaattcctaggaattcttgccaaacctacaggtggggtctttcattcccccctttttctggagactaaataaaatcttttattttatcgataagcttggctgcaggtcgacgcggccgc

SEQ ID NO: 9 - DNA, 310 bp, Homo sapiens
tttaaatactctcgacttgaaacccagagaggaatgtaagagcatccatcagcggtggtctcactgctcactggttcttgggatgtggatgggagaatgaagaagggctatcaggccagccttcagaggactccaaggaacattcaacgctgtcggtgagtttgggatttgaaaaaaccactgaccgttgactgtaccttggggtccttacagacgacactacatttcctgaagcaaaagagcaagctgtaccttcacatgtcacatgagttcaccagaaatggtcctgcaatcccccaaatgtggtcca

(19) United States
(12) Patent Application Publication
(10) Pub. No.: US 2021/0069245 A1
(43) Pub. Date: Mar. 11, 2021

(54) MIRNA MODULATION OF T CELL SIGNALING AND USES THEREOF

(71) Applicant: Achelois Oncology, Inc., Redwood City, CA (US)

(72) Inventors: Chang-Zheng CHEN, Palo Alto, CA (US); Cordelia YU, Palo Alto, CA (US); Tianqiang SUN, San Francisco, CA (US); Hanane LAKLAI, San Diego, CA (US)

(21) Appl. No.: 16/960,529

(22) PCT Filed: Jan. 7, 2019
(86) PCT No.: PCT/US2019/012546

§ 371 Date: Jul. 7, 2020

Related U.S. Application Data
(60) Provisional application No. 62/614,924, filed on Jan. 8, 2018.

(51) Int. Cl. A61K 35/17; C12N 15/11; A61P 35/00

(52) U.S. Cl. CPC A61K 35/17; A61P 35/00; C12N 15/111

(57) ABSTRACT

Provided are methods of treating cancer using adoptive cell therapy with T cells modified to have a reduced T cell receptor (TCR) signaling threshold and/or increased TCR sensitivity, and to have improved anti-tumor properties, such as increased cytotoxic activity and reduced susceptibility to immune suppression and/or exhaustion. Also provided are methods of making and compositions comprising such modified T cells.

### RELATED APPLICATIONS

The present application claims the benefit of, and priority to U.S. Provisional Application No. 62/614,924, filed Jan. 8, 2018. The content of the provisional application is incorporated herein by reference in its entirety.

### SUBMISSION OF SEQUENCE LISTING ON ASCII TEXT FILE

The content of the following submission on ASCH text file is incorporated herein by reference in its entirety: a computer readable form (CRF) of the Sequence Listing (file name: 756592000140SEQLIST.TXT, date recorded: Jan. 7, 2019, size: 3 KB).

### TECHNICAL FIELD

This invention pertains to methods of treating cancer using adoptive cell therapy with T cells modified to have a reduced T cell receptor (TCR) signaling threshold and/or increased TCR sensitivity, and to have improved anti-tumor properties, such as increased cytotoxic activity and reduced susceptibility to immune suppression and/or exhaustion, and includes methods of making and compositions comprising such modified T cells.

### BACKGROUND

T cell mediated immunity is an adaptive process involving the development of antigen-specific T lymphocytes capable of eliminating viral, bacterial, or parasitic infections, or malignant cells. Aberrant recognition of self-antigens by T cells can lead to autoimmune inflammatory diseases. The antigen specificity of T lymphocytes is based on recognition by the T cell receptor (TCR) of antigenic peptides presented by major histocompatibility complex (MHC) molecules on antigen-presenting cells (APCs) (Broere, et al., Principles of Immunopharmacology, 2011). Each T lymphocyte expresses a unique TCR on the cell surface as the result of developmental selection upon maturation in the thymus.

In the past two decades, fundamental advances in immunology and tumor biology, combined with the identification of a large number of tumor antigens, have led to significant progress in the field of cell-based immunotherapy. T cell therapy occupies a large space in the field of cell-based immunotherapy, with the goal of treating cancer by transferring autologous and ex vivo expanded T cells to patients, and has resulted in some notable antitumor responses (Blattman et al., Science. 305(5681):200-5, 2004). For example, the administration of naturally occurring tumor-infiltrating lymphocytes (TILs) expanded ex vivo mediated an objective response rate ranging from 50-70% in melanoma patients, including bulky invasive tumors at multiple sites involving liver, lung, soft tissue and brain (Rosenberg et al., Nat. Rev. Cancer. 8(4):299-308, 2008; Dudley M E et al., J. Clin. Oncol. 23(10):2346-57, 2005).

Tumor-infiltrating lymphocytes (TILs) and other anti-tumor T cells derived from hosts are known to have anti-tumor activities, but significant limitations have prevented the widespread application of TIL therapy for treating cancer. Since many tumor antigens are endogenous proteins, and our immune tolerance system is highly effective in eliminating T cells bearing TCRs with high affinity against self-antigens, TILs often have TCRs with low or medium affinity against tumor antigens. Moreover, their anti-tumor functions are usually sensitive to suppression in the tumor microenvironment. As an alternative approach, exogenous high-affinity receptors, including TCRs and chimeric antigen receptors (CARs), have be introduced into normal autologous T cells of the patients through T cell engineering. The adoptive transfer of these cells into lympho-depleted patients has been shown to mediate cancer regression in cancers such as melanoma, colorectal carcinoma, and synovial sarcoma (Kunert R et al., Front. Immunol. 4:363, 2013). A recent phase I clinical trial using anti NY-ESO-1 TCRs against synovial sarcoma reported an overall response rate of 66% and complete response was achieved in one of the patients receiving the T cell therapy (Robbins P F et al., Clin. Cancer Res. 21(5):1019-27, 2015).

Identification of target-specific TCRs requires the establishment of target peptide/MHC specific TCR clones from patient T cells and screening for the right α-β chain combination that has the optimal target antigen-binding affinity. In vivo affinity maturation is often employed after cloning of a TCR from patient T cells to further enhance the target binding affinity of the TCR. The whole process requires expertise in many areas and is time-consuming (Kobayashi E et al., Oncoimmunology. 3(1):e27258, 2014). The difficulties in the TCR discovery process have largely impeded the widespread application of TCR-modified T cell therapy. It has also been hampered by treatment-related toxicity, in particularly with TCRs against antigens that are over-expressed on tumor cells but also expressed on healthy cells, or with TCRs recognizing off-target peptide/MHC complexes (Rosenberg S A et al., Science. 348(6230):62-8, 2015).

CAR T cell therapy merges the exquisite targeting specificity of monoclonal antibodies with the potent cytotoxicity and long-term persistence provided by cytotoxic T cells. A CAR is generally composed of an extracellular domain that recognizes a cell surface antigen, a transmembrane region, and an intracellular signaling domain. Binding of target antigens by CARs grafted onto a T cell surface can trigger T cell effector functions independent of TCR-peptide/MHC complex interaction. Thus, T cells equipped with CARs can be redirected to attack a broad variety of cells, including those that do not match the MHC type of the TCRs on the T cells but express the target cell-surface antigens. This approach overcomes the constraints of MHC-restricted TCR recognition and avoids tumor escape through impairments in antigen presentation or MHC molecule expression. Clinical trials have shown clinically significant antitumor activity of CAR T cell therapy in neuroblastoma (Louis C U et al., Blood. 118(23):6050-6056, 2011), B-ALL (Maude, S L, et al., New England Journal of Medicine 371:16:1507-1517, 2014), CLL (Brentjens, R J, et al. Blood 118:18:4817-4828, 2011), and B cell lymphoma (Kochenderfer, J N, et al. Blood 116:20:4099-4102, 2010).

Despite these early successes, CAR T cell therapy faces several hurdles that must be overcome, including efficacy issues resulting from CAR design, relapse with escape variants, and survival in the tumor microenvironment, as well as safety issues resulting from the extreme potency of CAR-modified T cells, which can lead to life-threatening conditions such as cytokine release syndrome (CRS) and macrophage activation syndrome (MAS), as well as tumor lysis syndrome (TLS), on-target off-tumor toxicity, and neurotoxicity (Almisbak, H. et al., Journal of Immunology Research, 2016). More importantly, some of the intrinsic limitations may prevent the broad adoption of CAR T cell therapy in solid tumors. Discovering tumor-specific antigens that are amenable to CAR T cell targeting is challenging and time-consuming for solid tumors. Therefore, on-target/off-tumor toxicity is likely hard to avoid. The polyspecific antigen recognition even by monoclonal antibodies also presents challenges to predicting the off-target toxicity of CAR T cells. Finally, CAR T cells as monoclonal therapeutic agents are susceptible to antigen-escape as tumor cells evolve through mutagenesis.

Therefore, methods of boosting the reactivity of TILs against a multitude of tumor antigens may help overcome many of the intrinsic limitations of CAR-T and TCR-T technologies, and enable TIL therapy to be broadly applicable to solid tumors.

The disclosures of all publications, patents, patent applications and published patent applications referred to herein are hereby incorporated herein by reference in their entirety.

### SUMMARY

In some embodiments, there is provided a method of treating cancer in an individual, comprising administering to the individual a population of modified T cells that recognize a cancer-associated antigen, wherein the modified T cells comprise an exogenous nucleic acid molecule encoding an RNA transcript comprising a microRNA (miRNA) that comprises a seed sequence having the nucleotide sequence of SEQ ID NO: 1, and wherein the modified T cells have a lower T cell receptor (TCR) signaling threshold and/or increased TCR sensitivity to the cancer-associated antigen as compared to T cells not comprising the exogenous miRNA.

In some embodiments, according to any of the method of treating cancer described above, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6.

In some embodiments, according to any of the method of treating cancer described above, the RNA transcript comprises a sequence corresponding to a precursor miRNA (pre-miRNA). In some embodiments, the RNA transcript comprises a sequence corresponding to a primary miRNA (pri-miRNA). In some embodiments, the RNA transcript comprises a loop region having the nucleotide sequence of SEQ ID NO: 4 or 5, or a variant thereof comprising up to 3 nucleotide substitutions. In some embodiments, the RNA transcript comprises a loop region having the nucleotide sequence of SEQ ID NO: 4 or 5. In some embodiments, the RNA transcript comprises a stem region having the nucleotide sequence of SEQ ID NO: 3, or a variant thereof comprising up to 3 nucleotide substitutions. In some embodiments, the RNA transcript comprises a stem region having the nucleotide sequence of SEQ ID NO: 3. In some embodiments, the sequence corresponding to the pre-miRNA has the nucleotide sequence of SEQ ID NO: 6 or 7. In some embodiments, the sequence corresponding to the pri-miRNA has the nucleotide sequence of SEQ ID NO: 8 or 9.

In some embodiments, according to any of the method of treating cancer described above, the method further comprises introducing the exogenous nucleic acid molecule into a population of input T cells, thereby generating the population of modified T cells. In some embodiments, the exogenous nucleic acid molecule is introduced by viral transduction, transposition, electroporation, or chemical transfection.

In some embodiments, there is provided a method of treating cancer in an individual, comprising administering to the individual a population of modified T cells that recognize a cancer-associated antigen in the individual, wherein the modified T cells comprise a modification that increases the expression of endogenous miR-181a, and wherein the modified T cells have a lower T cell receptor (TCR) signaling threshold and/or increased TCR sensitivity to the cancer-associated antigen as compared to T cells not modified to increase the expression of endogenous miR-181a. In some embodiments, the modification comprises introducing a nucleic acid molecule encoding a) a fusion protein comprising a nuclease-deficient sequence-guided endonuclease (SGEN) fused to an activator of RNA polymerase II; and b) a guide nucleotide directing the fusion protein to the promoter region of the miR-181a gene. In some embodiments, the modification comprises inserting a nucleic acid sequence that upregulates miR-181a into the genome of the T cells. In some embodiments, the nucleic acid sequence encodes miR-181a. In some embodiments, the nucleic acid sequence comprises a promoter sequence and is inserted such that the promoter sequence is operably linked to the miR-181a gene. In some embodiments, the nucleic acid sequence is inserted by homologous recombination. In some embodiments, the nucleic acid sequence is inserted using CRISPR. In some embodiments, the nucleic acid sequence is inserted by random integration. In some embodiments, the nucleic acid sequence is inserted by viral transduction. In some embodiments, the modification comprises a modification to the miR-181a gene that leads to an increase in the stability of the mRNA transcript of the miR-181a gene. In some embodiments, the method further comprises modifying a population of input T cells, thereby generating the population of modified T cells.

In some embodiments, according to any of the method of treating cancer described above, the method further comprises administering a second therapy or therapeutic agent. In some embodiments, the method further comprises administering a conditioning chemotherapy prior to administration of the modified T cells. In some embodiments, the method further comprises administering a chemotherapeutic agent. In some embodiments, the method further comprises administering an immunotherapeutic agent. In some embodiments, the immunotherapeutic agent is selected from the group consisting of IL-2, IL-7, IL-15, IL-12 and IL-21.

In some embodiments, according to any of the method of treating cancer described above, the modified T cells are autologous to the individual. In some embodiments, the method further comprises isolating T cells from the individual, thereby generating the autologous input T cells. In some embodiments, the T cells are isolated from a solid tumor in the individual.

In some embodiments, according to any of the method of treating cancer described above, the modified T cells are allogeneic to the individual.

In some embodiments, according to any of the method of treating cancer described above, the dose of the modified T cells administered to the individual is at least about 1×10⁵ cells per kilogram body weight of the individual. In some embodiments, the dose of the modified T cells administered to the individual is at least about 1×10⁷ cells. In some embodiments, the dose of the modified T cells administered to the individual is at least about 1×10⁷ cells/m² body surface area of the individual.

In some embodiments, according to any of the method of treating cancer described above, the modified T cells are administered to the individual by intravenous, intraperitoneal, or subcutaneous injection.

In some embodiments, according to any of the method of treating cancer described above, the individual is human.

In some embodiments, according to any of the method of treating cancer described above, the cancer is a solid tumor. In some embodiments, the cancer is pancreatic cancer, breast cancer, or melanoma. In some embodiments, the cancer is metastatic cancer.

In some embodiments, there is provided a method of producing a population of modified T cells, comprising introducing into a population of input T cells recognizing a cancer-associated antigen an exogenous nucleic acid molecule encoding an miRNA that comprises a seed sequence having the nucleotide sequence of SEQ ID NO: 1, wherein the modified T cells have a lower T cell receptor (TCR) signaling threshold and/or increased TCR sensitivity to the cancer-associated antigen. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6.

In some embodiments, according to any of the methods of producing a population of modified T cells described above, the RNA transcript comprises a sequence corresponding to a precursor miRNA (pre-miRNA). In some embodiments, the RNA transcript comprises a sequence corresponding to a primary miRNA (pri-miRNA). In some embodiments, the RNA transcript comprises a loop region having the nucleotide sequence of SEQ ID NO: 4 or 5, or a variant thereof comprising up to 3 nucleotide substitutions. In some embodiments, the RNA transcript comprises a loop region having the nucleotide sequence of SEQ ID NO: 4 or 5. In some embodiments, the RNA transcript comprises a stem region having the nucleotide sequence of SEQ ID NO: 3, or a variant thereof comprising up to 3 nucleotide substitutions. In some embodiments, the RNA transcript comprises a stem region having the nucleotide sequence of SEQ ID NO: 3. In some embodiments, the sequence corresponding to the pre-miRNA has the nucleotide sequence of SEQ ID NO: 6 or 7. In some embodiments, the sequence corresponding to the pri-miRNA has the nucleotide sequence of SEQ ID NO: 8 or 9.

In some embodiments, according to any of the methods of producing a population of modified T cells described above, the exogenous nucleic acid molecule is introduced by viral transduction, transposition, electroporation, or chemical transfection.

In some embodiments, there is provided a method of producing a population of modified T cells, comprising introducing into a population of input T cells recognizing a cancer-associated antigen a modification that increases the expression of endogenous miR-181a, wherein the modified T cells have a lower TCR signaling threshold and/or increased TCR sensitivity to the cancer-associated antigen. In some embodiments, the modification comprises introducing a nucleic acid molecule encoding a) a fusion protein comprising a nuclease-deficient sequence-guided endonuclease (SGEN) fused to an activator of RNA polymerase H; and b) a guide nucleotide directing the fusion protein to the promoter region of the miR-181a gene. In some embodiments, the modification comprises inserting a nucleic acid sequence that upregulates miR-181a into the genome of the T cells. In some embodiments, the nucleic acid sequence encodes miR-181a. In some embodiments, the nucleic acid sequence comprises a promoter sequence and is inserted such that the promoter sequence is operably linked to the miR-181a gene. In some embodiments, the nucleic acid sequence is inserted by homologous recombination. In some embodiments, the nucleic acid sequence is inserted using CRISPR. In some embodiments, the nucleic acid sequence is inserted by random integration. In some embodiments, the nucleic acid sequence is inserted by viral transduction. In some embodiments, the modification comprises a modification to the miR-181a gene that leads to an increase in the stability of the mRNA transcript of the miR-181a gene.

In some embodiments, according to any of the methods of producing a population of modified T cells described above, the input T cells are isolated from a solid tumor in an individual.

In some embodiments, the method further comprises isolating T cells from the solid tumor, thereby generating the population of input T cells.

In some embodiments, there is provided a population of modified T cells prepared by any of the methods described above.

In some embodiments, there is provided a composition comprising any of the populations of modified T cells described above.

In some embodiments, there is provided a pharmaceutical composition comprising any of the populations of modified T cells described above and a pharmaceutically acceptable carrier.

In some embodiments, there is provided a polyclonal population of modified T cells that recognize two or more cancer-associated antigens in an individual, wherein the modified T cells comprise an exogenous nucleic acid molecule encoding a microRNA (miRNA) that comprises a seed sequence having the nucleotide sequence of SEQ ID NO: 1, and wherein the modified T cells have a lower T cell receptor (TCR) signaling threshold and/or increased TCR sensitivity to the cancer-associated antigen.

In some embodiments, there is provided a polyclonal population of modified T cells that recognize two or more cancer-associated antigens in an individual, wherein the modified T cells comprise a modification that increases the expression of endogenous miR-181a, and wherein the modified T cells have a lower T cell receptor (TCR) signaling threshold and/or increased TCR sensitivity to the cancer-associated antigen.

### BRIEF DESCRIPTION OF THE DRAWINGS

FIG. 1 shows tumor cell killing of KPC pancreatic cancer cells mediated by control TILs or TILs transduced to overexpress miR-181 (miR-181 TILs).

FIGS. 2A and 2B show tumor growth in KPC mice, a mouse model of pancreatic cancer, treated with either control TILs+IL-2 or miR-181 TILs+IL-2.

FIG. 3 shows a survival plot for KPC mice treated with either control TILs+IL-2 or miR-181 TILs+IL-2.

FIG. 4 shows a survival plot for KPC mice treated with (i) no treatment, (ii) chemo+IL-2, (iii) chemo+control TILs+IL-2, or (iv) chemo+miR-181 TILs+IL-2.

FIG. 5 shows tumor cell killing of B16F0 melanoma cells mediated by control TILs or TILs transduced to overexpress miR-181.

FIG. 6 shows tumor growth in syngeneic B16F0 mice, a mouse model of melanoma, treated with 1) mock conditioning chemotherapy+mock TIL injection, 2) conditioning chemotherapy+mock TIL injection, 3) conditioning chemotherapy+control TILs, or 4) conditioning chemotherapy+miR-181 TILs.

FIG. 7 shows a survival plot for syngeneic B16F0 mice treated with 1) conditioning chemotherapy+mock TIL injection, 2) conditioning chemotherapy+control TILs, or 3) conditioning chemotherapy+miR-181 TILs.

FIG. 8 shows tumor cell killing of 4T1 mammary tumor cells mediated by control TILs or TILs transduced to overexpress miR-181.

FIG. 9 shows a survival plot for syngeneic 4T1 mice treated with 1) mock TIL injection, 2) control TILs, or 3) miR-181 TILs.

FIG. 10 shows the results of FACS analysis for PD-1 expression in vector control (409) TILs and miR-181 TILs sensitized to a gp100 peptide and re-challenged with either a non-specific peptide or the gp100 peptide.

FIG. 11 shows the results of FACS analysis for CD28 expression in vector control (409) TILs and miR-181 TILs sensitized to a gp100 peptide and re-challenged with either a non-specific peptide or the gp100 peptide.

### DETAILED DESCRIPTION

The original discovery of miR-181 function as a T cell sensitivity rheostat in controlling TCR signaling strength and T cell sensitivity illustrates the potential of using miR-181 to boost the reactivity of TILs against tumor antigens (Li, Qi-Jing, et al. Cell 129.1 (2007): 147-161; U.S. Pat. No. 9,364,522). However, it is unclear whether genetically modified TILs with ectopically expressed miR-181 may potentiate the anti-tumor function of TILs in vivo. It is important to note that the anti-tumor function of TILs depends not only on the strength of TCR signaling against tumor-antigens but also on other anti-tumor properties. For example, it is not known how miR-181 modification of TILs would affect their CTL activity against tumor cells, their proliferation, differentiation, and migration in tumors, and their sensitivity to the immune suppressive tumor microenvironment. The present application is based, at least in part, on the unexpected finding that modifying TILs to increase miR-181 expression can boost their anti-tumor activity in preclinical melanoma, breast, and pancreatic cancer models. Moreover, increased miR-181 expression provided many other beneficial effects on the anti-tumor function of TILs. Increased miR-181 expression boosted the CTL activity of TILs against tumor cells, suppressed the expression of PD-1 inhibitory checkpoint expression on TILs, and potentiated the expression of co-stimulatory molecule CD28. These findings for the first time provide evidence of the role of increased miR-181 in cancer therapy.

Provided herein are methods of treating cancer in an individual using adoptive cell therapy with a population of cancer-associated antigen-specific T cells having natural receptors and modified to have a reduced T cell receptor (TCR) signaling threshold and/or increased TCR sensitivity. In some embodiments, the modified T cells also have reduced susceptibility to immune suppression and/or exhaustion and increased CTL activity, thereby overcoming additional significant limitations of T cell therapy, immunosuppression and anergy. In some embodiments, the population of modified T cells is derived from a polyclonal population of input T cells, such as autologous T cells isolated from a solid tumor in the individual, and is capable of recognizing a plurality of cancer-associated antigens expressed by cancer cells in the individual, thus rendering therapy utilizing such cells less susceptible to relapse resulting from escape variants. In some embodiments, the population of modified T cells is derived from a polyclonal population of input T cells, such as anti-tumor T cells expanded in vino from autologous T cells with antigen presenting cells loaded with tumor antigens. In some embodiments, the population of modified T cells is derived from monoclonal T cells with specific TCRs recognizing a tumor-antigen. Also provided are methods of making the population of modified T cells, and compositions and articles of manufacture comprising the population of modified T cells.

### Definitions

As used herein, an “individual” is a mammal, such as a human or other animal. In some embodiments, the individual, e.g., patient, to whom the cells, cell populations, or compositions are administered is a mammal, e.g., a primate, such as a human. In some embodiments, the primate is a monkey or an ape. The individual can be male or female and can be any suitable age, including infant, juvenile, adolescent, adult, and geriatric individuals. In some embodiments, the individual is a non-primate mammal, such as a rodent.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply necessarily complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated.

As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in an individual that may be predisposed to the disease but has not yet been diagnosed with the disease. In some embodiments, the provided cells and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, cells that suppress tumor growth reduce the rate of growth of the tumor compared to the rate of growth of the tumor in the absence of the cells.

An “effective amount” of an agent, e.g., a pharmaceutical formulation, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the cells and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount. In the context of lower tumor burden, the prophylactically effective amount in some aspects will be higher than the therapeutically effective amount.

### Methods of Treatment

Provided are methods of administering the cells, populations, and compositions described herein, and uses of such cells, populations, and compositions to treat or prevent diseases, conditions, and disorders, such as cancer. In some embodiments, the cells, populations, and compositions are administered to an individual or patient having the particular disease or condition to be treated, e.g., via targeting of disease cells (such as cancer cells) by the modified T cells. In some embodiments, cells and compositions prepared by the provided methods, such as engineered compositions and end-of-production compositions following incubation and/or other processing steps, are administered to an individual, such as an individual having or at risk for the disease or condition. In some aspects, the methods thereby treat, e.g., ameliorate one or more symptom of, the disease or condition, such as by lessening tumor burden in a cancer expressing an antigen recognized by the modified T cell.

In some embodiments, the provided methods generally involve administering doses of the provided modified T cells to individuals having cancer, such as a cancer a component of which is specifically recognized by the modified T cell. The administrations generally effect an improvement in one or more symptoms of the cancer and/or treat or prevent the cancer or symptoms thereof. In some embodiments, a T cell growth factor that promotes the growth and activation of the modified T cells is administered to the individual either concomitantly with the modified T cells or subsequently to the modified T cells. The T cell growth factor can be any suitable growth factor that promotes the growth and activation of the modified T cells. Examples of suitable T cell growth factors include interleukin (IL)-2, IL-7, IL-15, IL-12 and IL-21, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

In some embodiments, prior to administering the modified T cells into the individual, the individual is lymphodepleted, for example, using a cocktail of drugs to deplete T and B cells. In some embodiments, prior to administering the modified T cells into the individual, a conditioning chemotherapy is administered to the individual. In some embodiments, the conditioning chemotherapy comprises cyclophosphamide and fludarabine. In some embodiments, IL-2 is systemically administered in order to help support the transferred modified T cells following lymphodepletion and cell infusion. Treatment with IL-2 after T cell infusion supports the persistence of the infused T cells in vivo.

Among the cancers to be treated are tumors, including solid tumors, hematologic malignancies, and melanomas. Such cancers include but are not limited to solid tumors including sarcomas and carcinomas, including adrenocortical carcinoma, cholangiocarcinoma, fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, stomach cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, thyroid cancer (e.g., medullary thyroid carcinoma and papillary thyroid carcinoma), pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), colorectal cancer, cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, esophageal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, kidney cancer, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, plasmacytoma, Waldenstrom's macroglobulinemia, heavy chain disease, myelodysplastic syndrome, hairy cell leukemia and myelodysplasia.

In some embodiments, according to any of the methods of treating cancer described herein, the cancer is pancreatic cancer. In some embodiments, the pancreatic cancer is pancreatic exocrine cancer. In some embodiments, the pancreatic cancer is pancreatic neuroendocrine cancer.

In some embodiments, according to any of the methods of treating cancer described herein, the cancer is pancreatic exocrine cancer. In some embodiments, the pancreatic exocrine cancer is pancreatic adenocarcinoma (such as invasive or ductal pancreatic adenocarcinoma), acinar cell carcinoma of the pancreas, cystadenocarcinoma, pancreatoblastoma, or pancreatic mucinous cystic neoplasm. In some embodiments, the individual may be a human who has a gene, genetic mutation, or polymorphism associated with pancreatic exocrine cancer or has one or more extra copies of a gene associated with pancreatic neuroendocrine cancer.

In some embodiments, according to any of the methods of treating cancer described herein, the cancer is pancreatic neuroendocrine cancer. In some embodiments, the pancreatic neuroendocrine cancer is a well-differentiated neuroendocrine tumor, a well-differentiated (low grade) neuroendocrine carcinoma, or a poorly differentiated (high grade) neuroendocrine carcinoma. In some embodiments, the pancreatic neuroendocrine cancer is a functional pancreatic neuroendocrine tumor. In some embodiments, the pancreatic neuroendocrine tumor is a nonfunctional pancreatic neuroendocrine tumor. In some embodiments, the pancreatic neuroendocrine cancer is insulinoma, glucagonoma, somatostatinoma, gastrinoma, VIPoma, GRFoma, or ACTHoma. In some embodiments, the individual may be a human who has a gene, genetic mutation, or polymorphism associated with pancreatic neuroendocrine cancer (e.g., NF1 and/or MEN1) or has one or more extra copies of a gene associated with pancreatic neuroendocrine cancer.

In some embodiments, according to any of the methods of treating cancer described herein, the cancer is breast cancer. In some embodiments, the breast cancer is early stage breast cancer, non-metastatic breast cancer, advanced breast cancer, stage IV breast cancer, locally advanced breast cancer, metastatic breast cancer, breast cancer in remission, breast cancer in an adjuvant setting, or breast cancer in a neoadjuvant setting. In some embodiments, the breast cancer is in a neoadjuvant setting. In some embodiments, the breast cancer is at an advanced stage. In some embodiments, the breast cancer (which may be HER2 positive or HER2 negative) includes, for example, advanced breast cancer, stage IV breast cancer, locally advanced breast cancer, and metastatic breast cancer. In some embodiments, the individual may be a human who has a gene, genetic mutation, or polymorphism associated with breast cancer (e.g., BRCA1, BRCA2, ATM, CHEK2, RAD51, AR, DIRAS3, ERBB2, TP53, AKT, PTEN, and/or PDK) or has one or more extra copies of a gene (e.g., one or more extra copies of the HER2 gene) associated with breast cancer. In some embodiments, the method further comprises identifying a cancer patient population (i.e. breast cancer population) based on a hormone receptor status of patients having tumor tissue not expressing both ER and PgR.

In some embodiments, according to any of the methods of treating cancer described herein, the cancer is melanoma. In some embodiments, the melanoma is superficial spreading melanoma, lentigo maligna melanoma, nodular melanoma, mucosal melanoma, polypoid melanoma, desmoplastic melanoma, amelanotic melanoma, soft-tissue melanoma, or acral lentiginous melanoma. In some embodiments, the melanoma is at any of stage I, II, II, or IV, according to the American Joint Committee on Cancer (AJCC) staging groups. In some embodiments, the melanoma is recurrent.

Methods for administration of cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338.

In some embodiments, the cell therapy, e.g., adoptive T cell therapy, is carried out by autologous transfer, in which the cells are isolated and/or otherwise prepared from the individual who is to receive the cell therapy, or from a sample derived from such an individual. Thus, in some aspects, the cells are derived from an individual, e.g., patient, in need of a treatment and the cells, following isolation and processing are administered to the same individual.

In some embodiments, the cell therapy, e.g., adoptive T cell therapy, is carried out by allogeneic transfer, in which the cells are isolated and/or otherwise prepared from an individual other than an individual who is to receive or who ultimately receives the cell therapy, e.g., a first individual. In such embodiments, the cells then are administered to a different individual, e.g., a second individual, of the same species. In some embodiments, the first and second individuals are genetically identical or similar. In some embodiments, the second individual expresses the same HLA class or supertype as the first individual.

The cells can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, intrathoracic, intracranial, or subcutaneous administration.

In some embodiments, a given dose is administered by a single bolus administration of the cells. In some embodiments, it is administered by multiple bolus administrations of the cells, for example, over a period of no more than 3 days, or by continuous infusion administration of the cells.

For the prevention or treatment of disease, the appropriate dosage may depend on the type of disease to be treated, the type of cells or target antigens, the severity and course of the disease, whether the cells are administered for preventive or therapeutic purposes, previous therapy, the individual's clinical history and response to the cells, and the discretion of the attending physician. The compositions and cells are in some embodiments suitably administered to the individual at one time or over a series of treatments.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

In some embodiments, a single administration of the modified T cells or composition to the individual results in an improved clinical endpoint (e.g., overall survival, progression-free survival, time to progression, time to treatment failure, event-free survival, time to next treatment, objective response rate, or duration of response) in the individual compared to the clinical endpoint resulting from a comparable single administration of the input T cells or composition comprising such input T cells to the individual. In some embodiments, the increase is at least 1.2-fold, 1.5-fold, 2-fold, 3-fold, 4-fold, or 5-fold.

In some embodiments, the method results in a duration of response (time from documentation of tumor response to disease progression) in an individual of at least about 1 month, at least about 2 months, at least about 6 months, at least about a year, at least about 2 years, or more. In some embodiments, a single administration of the modified T cells or composition to the individual results in an increased duration of response in the subject compared to the duration of response resulting from a comparable single administration of the input T cells or composition comprising such input T cells to the individual. In some embodiments, the increase is at least 1.2-fold, 1.5-fold, 2-fold, 3-fold, 4-fold, or 5-fold.

In some embodiments, there is provided a method of treating cancer in an individual, comprising administering to the individual a population of modified T cells that recognize a cancer-associated antigen, wherein the modified T cells comprise an exogenous nucleic acid molecule encoding an RNA transcript comprising an miRNA, wherein the miRNA comprises a seed sequence having the nucleotide sequence of SEQ ID NO: 1, and wherein the modified T cells have a lower T cell receptor (TCR) signaling threshold and/or increased TCR sensitivity to the cancer-associated antigen as compared to T cells not comprising the exogenous miRNA. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the RNA transcript comprises a loop region having the nucleotide sequence of SEQ ID NO: 4, or a variant thereof comprising up to 3 nucleotide substitutions. In some embodiments, the RNA transcript comprises a loop region having the nucleotide sequence of SEQ ID NO: 4. In some embodiments, the RNA transcript comprises a stem region having the nucleotide sequence of SEQ ID NO: 3, or a variant thereof comprising up to 3 nucleotide substitutions. In some embodiments, the RNA transcript comprises a stem region having the nucleotide sequence of SEQ ID NO: 3. In some embodiments, the RNA transcript comprises the sequence of SEQ ID NO: 2. In some embodiments, the RNA transcript comprises a sequence corresponding to a precursor miRNA (pre-miRNA). In some embodiments, the sequence corresponding to the pre-miRNA has the nucleotide sequence of SEQ ID NO: 6 or 7. In some embodiments, the RNA transcript comprises a sequence corresponding to a primary miRNA (pri-miRNA). In some embodiments, the sequence corresponding to the pri-miRNA has the nucleotide sequence of SEQ ID NO: 8 or 9. In some embodiments, the method further comprises introducing the exogenous nucleic acid molecule into a population of input T cells, thereby generating the population of modified T cells. In some embodiments, the exogenous nucleic acid molecule is introduced by viral transduction, transposition, electroporation, or chemical transfection. In some embodiments, the modified T cells are autologous to the individual. In some embodiments, the method further comprises isolating T cells from the individual, thereby generating the population of input T cells. In some embodiments, the T cells are isolated from a solid tumor in the individual. In some embodiments, the modified T cells are allogeneic to the individual. In some embodiments, the dose of the modified T cells administered to the individual is at least about 1×10⁵ cells per kilogram body weight of the individual. In some embodiments, the dose of the modified T cells administered to the individual is at least about 1×10⁷ cells. In some embodiments, the dose of the modified T cells administered to the individual is at least about 1×10⁷ cells/m² body surface area of the individual. In some embodiments, the modified T cells are administered to the individual by intravenous, intraperitoneal, or subcutaneous injection. In some embodiments, the method further comprises administering a conditioning chemotherapy to the individual prior to administration of the modified T cells. In some embodiments, the conditioning chemotherapy comprises cyclophosphamide and fludarabine. In some embodiments, the method further comprises administering to the individual a suitable growth factor that promotes the growth and activation of the modified T cells, including, e.g., IL-2, IL-7, IL-15, IL-12 and IL-21. In some embodiments, the method further comprises administering IL-2 to the individual. In some embodiments, the IL-2 is administered to the individual concomitantly with the modified T cells and/or subsequently to the modified T cells. In some embodiments, the cancer is a solid tumor. In some embodiments, the cancer is pancreatic cancer, breast cancer, or melanoma. In some embodiments, the cancer is metastatic cancer. In some embodiments, the individual is human.

In some embodiments, there is provided a method of treating pancreatic cancer in an individual, comprising administering to the individual a population of modified T cells that recognize a pancreatic cancer-associated antigen, wherein the modified T cells comprise an exogenous nucleic acid molecule encoding an RNA transcript comprising an miRNA, wherein the miRNA comprises a seed sequence having the nucleotide sequence of SEQ ID NO: 1, and wherein the modified T cells have a lower T cell receptor (TCR) signaling threshold and/or increased TCR sensitivity to the pancreatic cancer-associated antigen. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the RNA transcript comprises a loop region having the nucleotide sequence of SEQ ID NO: 4, or a variant thereof comprising up to 3 nucleotide substitutions. In some embodiments, the RNA transcript comprises a loop region having the nucleotide sequence of SEQ ID NO: 4. In some embodiments, the RNA transcript comprises a stem region having the nucleotide sequence of SEQ ID NO: 3, or a variant thereof comprising up to 3 nucleotide substitutions. In some embodiments, the RNA transcript comprises a stem region having the nucleotide sequence of SEQ ID NO: 3. In some embodiments, the RNA transcript comprises the sequence of SEQ ID NO: 2. In some embodiments, the RNA transcript comprises a sequence corresponding to a precursor miRNA (pre-miRNA). In some embodiments, the sequence corresponding to the pre-miRNA has the nucleotide sequence of SEQ ID NO: 6 or 7. In some embodiments, the RNA transcript comprises a sequence corresponding to a primary miRNA (pri-miRNA). In some embodiments, the sequence corresponding to the pri-miRNA has the nucleotide sequence of SEQ ID NO: 8 or 9. In some embodiments, the method further comprises introducing the exogenous nucleic acid molecule into a population of input T cells, thereby generating the population of modified T cells. In some embodiments, the exogenous nucleic acid molecule is introduced by viral transduction, transposition, electroporation, or chemical transfection. In some embodiments, the modified T cells are autologous to the individual. In some embodiments, the method further comprises isolating T cells from the individual, thereby generating the population of input T cells. In some embodiments, the T cells are isolated from a solid tumor in the individual. In some embodiments, the modified T cells are allogeneic to the individual. In some embodiments, the dose of the modified T cells administered to the individual is at least about 1×10⁵ cells per kilogram body weight of the individual. In some embodiments, the dose of the modified T cells administered to the individual is at least about 1×10⁷ cells. In some embodiments, the dose of the modified T cells administered to the individual is at least about 1×10⁷ cells/m² body surface area of the individual. In some embodiments, the modified T cells are administered to the individual by intravenous, intraperitoneal, or subcutaneous injection. In some embodiments, the method further comprises administering a conditioning chemotherapy to the individual prior to administration of the modified T cells. In some embodiments, the conditioning chemotherapy comprises cyclophosphamide and fludarabine. In some embodiments, the method further comprises administering to the individual a suitable growth factor that promotes the growth and activation of the modified T cells, including, e.g., IL-2, IL-7, IL-15, IL-12 and IL-21. In some embodiments, the method further comprises administering IL-2 to the individual. In some embodiments, the IL-2 is administered to the individual concomitantly with the modified T cells and/or subsequently to the modified T cells. In some embodiments, the pancreatic cancer is metastatic pancreatic cancer. In some embodiments, the individual is human.

In some embodiments, there is provided a method of treating breast cancer in an individual, comprising administering to the individual a population of modified T cells that recognize a breast cancer-associated antigen, wherein the modified T cells comprise an exogenous nucleic acid molecule encoding an RNA transcript comprising an miRNA, wherein the miRNA comprises a seed sequence having the nucleotide sequence of SEQ ID NO: 1, and wherein the modified T cells have a lower T cell receptor (TCR) signaling threshold and/or increased TCR sensitivity to the breast cancer-associated antigen. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the RNA transcript comprises a loop region having the nucleotide sequence of SEQ ID NO: 4, or a variant thereof comprising up to 3 nucleotide substitutions. In some embodiments, the RNA transcript comprises a loop region having the nucleotide sequence of SEQ ID NO: 4. In some embodiments, the RNA transcript comprises a stem region having the nucleotide sequence of SEQ ID NO: 3, or a variant thereof comprising up to 3 nucleotide substitutions. In some embodiments, the RNA transcript comprises a stem region having the nucleotide sequence of SEQ ID NO: 3. In some embodiments, the RNA transcript comprises the sequence of SEQ ID NO: 2. In some embodiments, the RNA transcript comprises a sequence corresponding to a precursor miRNA (pre-miRNA). In some embodiments, the sequence corresponding to the pre-miRNA has the nucleotide sequence of SEQ ID NO: 6 or 7. In some embodiments, the RNA transcript comprises a sequence corresponding to a primary miRNA (pri-miRNA). In some embodiments, the sequence corresponding to the pri-miRNA has the nucleotide sequence of SEQ ID NO: 8 or 9. In some embodiments, the method further comprises introducing the exogenous nucleic acid molecule into a population of input T cells, thereby generating the population of modified T cells. In some embodiments, the exogenous nucleic acid molecule is introduced by viral transduction, transposition, electroporation, or chemical transfection. In some embodiments, the modified T cells are autologous to the individual. In some embodiments, the method further comprises isolating T cells from the individual, thereby generating the population of input T cells. In some embodiments, the T cells are isolated from a solid tumor in the individual. In some embodiments, the modified T cells are allogeneic to the individual. In some embodiments, the dose of the modified T cells administered to the individual is at least about 1×10⁵ cells per kilogram body weight of the individual. In some embodiments, the dose of the modified T cells administered to the individual is at least about 1×10⁷ cells. In some embodiments, the dose of the modified T cells administered to the individual is at least about 1×10⁷ cells/m² body surface area of the individual. In some embodiments, the modified T cells are administered to the individual by intravenous, intraperitoneal, or subcutaneous injection. In some embodiments, the method further comprises administering a conditioning chemotherapy to the individual prior to administration of the modified T cells. In some embodiments, the conditioning chemotherapy comprises cyclophosphamide and fludarabine. In some embodiments, the method further comprises administering to the individual a suitable growth factor that promotes the growth and activation of the modified T cells, including, e.g., IL-2, IL-7, IL-15, IL-12 and IL-21. In some embodiments, the method further comprises administering IL-2 to the individual. In some embodiments, the IL-2 is administered to the individual concomitantly with the modified T cells and/or subsequently to the modified T cells. In some embodiments, the breast cancer is metastatic breast cancer. In some embodiments, the individual is human.

In some embodiments, there is provided a method of treating melanoma in an individual, comprising administering to the individual a population of modified T cells that recognize a melanoma-associated antigen, wherein the modified T cells comprise an exogenous nucleic acid molecule encoding an RNA transcript comprising an miRNA, wherein the miRNA comprises a seed sequence having the nucleotide sequence of SEQ ID NO: 1, and wherein the modified T cells have a lower T cell receptor (TCR) signaling threshold and/or increased TCR sensitivity to the melanoma-associated antigen. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the RNA transcript comprises a loop region having the nucleotide sequence of SEQ ID NO: 4, or a variant thereof comprising up to 3 nucleotide substitutions. In some embodiments, the RNA transcript comprises a loop region having the nucleotide sequence of SEQ ID NO: 4. In some embodiments, the RNA transcript comprises a stem region having the nucleotide sequence of SEQ ID NO: 3, or a variant thereof comprising up to 3 nucleotide substitutions. In some embodiments, the RNA transcript comprises a stem region having the nucleotide sequence of SEQ ID NO: 3. In some embodiments, the RNA transcript comprises the sequence of SEQ ID NO: 2. In some embodiments, the RNA transcript comprises a sequence corresponding to a precursor miRNA (pre-miRNA). In some embodiments, the sequence corresponding to the pre-miRNA has the nucleotide sequence of SEQ ID NO: 6 or 7. In some embodiments, the RNA transcript comprises a sequence corresponding to a primary miRNA (pri-miRNA). In some embodiments, the sequence corresponding to the pri-miRNA has the nucleotide sequence of SEQ ID NO: 8 or 9. In some embodiments, the method further comprises introducing the exogenous nucleic acid molecule into a population of input T cells, thereby generating the population of modified T cells. In some embodiments, the exogenous nucleic acid molecule is introduced by viral transduction, transposition, electroporation, or chemical transfection. In some embodiments, the modified T cells are autologous to the individual. In some embodiments, the method further comprises isolating T cells from the individual, thereby generating the population of input T cells. In some embodiments, the T cells are isolated from a solid tumor in the individual. In some embodiments, the modified T cells are allogeneic to the individual. In some embodiments, the dose of the modified T cells administered to the individual is at least about 1×10⁵ cells per kilogram body weight of the individual. In some embodiments, the dose of the modified T cells administered to the individual is at least about 1×10⁷ cells. In some embodiments, the dose of the modified T cells administered to the individual is at least about 1×10⁷ cells/m² body surface area of the individual. In some embodiments, the modified T cells are administered to the individual by intravenous, intraperitoneal, or subcutaneous injection. In some embodiments, the method further comprises administering a conditioning chemotherapy to the individual prior to administration of the modified T cells. In some embodiments, the conditioning chemotherapy comprises cyclophosphamide and fludarabine. In some embodiments, the method further comprises administering to the individual a suitable growth factor that promotes the growth and activation of the modified T cells, including, e.g., IL-2, IL-7, IL-15, IL-12 and IL-21. In some embodiments, the method further comprises administering IL-2 to the individual. In some embodiments, the IL-2 is administered to the individual concomitantly with the modified T cells and/or subsequently to the modified T cells. In some embodiments, the melanoma is metastatic melanoma. In some embodiments, the individual is human.

In some embodiments, there is provided a method of treating cancer in an individual, comprising administering to the individual a population of modified T cells that recognize a cancer-associated antigen in the individual, wherein the modified T cells comprise a modification that increases the expression of endogenous miR-181a, and wherein the modified T cells have a lower T cell receptor (TCR) signaling threshold and/or increased TCR sensitivity to the cancer-associated antigen as compared to T cells not comprising the exogenous miRNA. In some embodiments, the modification comprises introducing a nucleic acid molecule encoding a) a fusion protein comprising a nuclease-deficient sequence-guided endonuclease (SGEN) fused to an activator of RNA polymerase H; and b) a guide nucleotide directing the fusion protein to the promoter region of a miR-181a gene. In some embodiments, the miR-181a gene is miR-181a-1. In some embodiments, the miR-181a gene is miR-181a-2. In some embodiments, the modification comprises introducing a nucleic acid sequence that upregulates miR-181a into the genome of the T cells. In some embodiments, the nucleic acid sequence encodes miR-181a. In some embodiments, the nucleic acid sequence comprises a promoter sequence and is inserted such that the promoter sequence is operably linked to an miR-181a gene (miR-181a-1 or miR-181a-2). In some embodiments, the nucleic acid sequence is inserted by homologous recombination. In some embodiments, the nucleic acid sequence is inserted using CRISPR In some embodiments, the nucleic acid sequence is inserted by random integration. In some embodiments, the nucleic acid sequence is inserted by viral transduction. In some embodiments, the modification comprises a modification to an miR-181a gene that leads to an increase in the stability of the mRNA transcript of the miR-181a gene. In some embodiments, the method further comprises introducing the modification into a population of input T cells, thereby generating the population of modified T cells. In some embodiments, the modified T cells are autologous to the individual. In some embodiments, the method further comprises isolating T cells from the individual, thereby generating the population of input T cells. In some embodiments, the T cells are isolated from a solid tumor in the individual. In some embodiments, the modified T cells are allogeneic to the individual. In some embodiments, the dose of the modified T cells administered to the individual is at least about 1×10⁵ cells per kilogram body weight of the individual. In some embodiments, the dose of the modified T cells administered to the individual is at least about 1×10⁷ cells. In some embodiments, the dose of the modified T cells administered to the individual is at least about 1×10⁷ cells/m² body surface area of the individual. In some embodiments, the modified T cells are administered to the individual by intravenous, intraperitoneal, or subcutaneous injection. In some embodiments, the method further comprises administering a conditioning chemotherapy to the individual prior to administration of the modified T cells. In some embodiments, the conditioning chemotherapy comprises cyclophosphamide and fludarabine. In some embodiments, the method further comprises administering to the individual a suitable growth factor that promotes the growth and activation of the modified T cells, including, e.g., IL-2, IL-7, IL-15, IL-12 and IL-21. In some embodiments, the method further comprises administering IL-2 to the individual. In some embodiments, the IL-2 is administered to the individual concomitantly with the modified T cells and/or subsequently to the modified T cells. In some embodiments, the cancer is a solid tumor. In some embodiments, the cancer is pancreatic cancer, breast cancer, or melanoma. In some embodiments, the cancer is metastatic cancer. In some embodiments, the individual is human.

### Combination Therapy

In some embodiments, the cells are administered as part of a combination treatment, such as simultaneously with or sequentially with, in any order, another therapeutic intervention, such as an antibody or engineered cell or receptor or other agent, such as a cytotoxic or therapeutic agent. Thus, the cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents.

In some embodiments, the methods comprise administration to the individual of a chemotherapeutic agent, e.g., a conditioning chemotherapeutic agent, for example, to reduce tumor burden prior to the dose administrations. In some embodiments, the methods comprise administration to the individual of a conditioning chemotherapy regimen prior to administration of the modified T cells. In some embodiments, a conditioning chemotherapy regimen according to any of the conditioning chemotherapy regiments known in the art is administered. For example, in some embodiments, the conditioning chemotherapy regimen comprises administration of cyclophosphamide and fludarabine.

In some embodiments, a method of treating cancer according to any of the embodiments described herein further comprises administering to the individual a second therapeutic agent. In some embodiments, the second therapeutic agent is a chemotherapeutic agent. In some embodiments, the second therapeutic agent is an immunotherapeutic agent. In some embodiments, the immunotherapeutic agent is selected from the group consisting of IL-2, IL-7, IL-15, IL-12 and IL-21.

### Dosing

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺ to CD8⁺ ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺ T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺ to CD8⁺ ratio), e.g., within a certain tolerated difference or error of such a ratio.

In some embodiments, the cells are administered at or within a tolerated difference of a desired dose of one or more of the individual populations or sub-types of cells, such as a desired dose of CD4+ cells and/or a desired dose of CD8+ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or sub-type, or minimum number of cells of the population or sub-type per unit of body weight.

Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺ and/or CD8⁺ cells/kg body weight.

In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8+ cells, and/or at least about 1×10⁶, about 2.5×10⁶, about 5×10⁶, about 7.5×10⁶, or about 9×10⁶ T cells. In some embodiments, the cells are administered at or within a certain range of error of between about 10⁸ and 10¹² or between about 10¹⁰ and 10¹¹ T cells, between about 10⁸ and 10¹² or between about 10¹⁰ and 10¹¹ CD4⁺ cells, and/or between about 10⁸ and 10¹² or between about 10¹⁰ and 10¹¹ CD8⁺ cells.

In some embodiments, the cells are administered at or within a tolerated range of a desired output ratio of multiple cell populations or sub-types, such as CD4+ and CD8+ cells or sub-types. In some aspects, the desired ratio can be a specific ratio or can be a range of ratios. for example, in some embodiments, the desired ratio (e.g., ratio of CD4⁺ to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In the context of adoptive cell therapy, administration of a given “dose” encompasses administration of the given amount or number of cells as a single composition and/or single uninterrupted administration, e.g., as a single injection or continuous infusion, and also encompasses administration of the given amount or number of cells as a split dose, provided in multiple individual compositions or infusions, over a specified period of time, which is no more than 3 days. Thus, in some contexts, the dose is a single or continuous administration of the specified number of cells, given or initiated at a single point in time. In some contexts, however, the dose is administered in multiple injections or infusions over a period of several days, such as no more than three days, such as once a day for three days or for two days or by multiple infusions over a single day period.

Thus, in some aspects, the cells are administered in a single pharmaceutical composition.

In some embodiments, the cells are administered in a plurality of compositions, collectively containing the cells of a single dose.

Thus, one or more of the doses in some aspects may be administered as a split dose. For example, in some embodiments, the dose may be administered to the subject over 2 days or over 3 days. Exemplary methods for split dosing include administering 25% of the dose on the first day and administering the remaining 75% of the dose on the second day. In other embodiments 33% of the dose may be administered on the first day and the remaining 67% administered on the second day. In some aspects, 10% of the dose is administered on the first day, 30% of the dose is administered on the second day, and 60% of the dose is administered on the third day. In some embodiments, the split dose is not spread over more than 3 days.

In some embodiments, multiple doses are given, e.g., by administering a first dose and one or more subsequent doses, with each subsequent dose given at a point in time that is greater than about 28 days after the administration of the first or prior dose.

In some embodiments, the dose contains a number of cells, number of modified T cells, or number of tumor-infiltrating lymphocytes (TILs) in the range from about 10⁵ to about 10⁶ of such cells per kilogram body weight of the subject, and/or a number of such cells that is no more than about 10⁵ or about 10⁶ such cells per kilogram body weight of the subject. For example, in some embodiments, the first or subsequent dose includes less than or no more than at or about 1×10⁵, at or about 2×10⁵, at or about 5×10⁵, or at or about 1×10⁶ of such cells per kilogram body weight of the subject. In some embodiments, the first dose includes at or about 1×10⁵, at or about 2×10⁵, at or about 5×10⁵, or at or about 1×10⁶ of such cells per kilogram body weight of the subject, or a value within the range between any two of the foregoing values. In particular embodiments, the numbers and/or concentrations of cells refer to the number of modified T cells. In other embodiments, the numbers and/or concentrations of cells refer to the number or concentration of all cells, T cells, or TILs administered.

In some embodiments, for example, where the subject is a human, the dose includes fewer than about 1×10⁸ total modified T cells, T cells, or TILs, e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the dose contains fewer than about 1×10⁸ total modified T cells, T cells, or TILs cells per m² of the subject, e.g., in the range of about 1×10⁶ to 1×10⁸ such cells per m² of the subject, such as 2×10⁶, 5×10⁴, 1×10⁷, 5×10⁷, or 1×10⁸ such cells per m of the subject, or the range between any two of the foregoing values.

In certain embodiments, the number of cells, modified T cells, T cells, or TILs in the dose is greater than about 1×10⁶ such cells per kilogram body weight of the subject, e.g., 2×10⁶, 3×10⁶, 5×10⁶, 1×10⁷, 5×10⁷, 1×10⁸, 1×10⁹, or 1×10¹⁰ such cells per kilogram of body weight and/or, 1×10⁸, or 1×10⁹, 1×10¹⁰ such cells per m² of the subject or total, or the range between any two of the foregoing values.

In some aspects, the size of the dose is determined based on one or more criteria such as response of the subject to prior treatment, e.g. chemotherapy, disease burden in the subject, such as tumor load, bulk, size, or degree, extent, or type of metastasis, stage, and/or likelihood or incidence of the subject developing toxic outcomes, e.g., CRS, macrophage activation syndrome, tumor lysis syndrome, neurotoxicity, and/or a host immune response against the cells and/or recombinant receptors being administered.

miRNA-Mediated Modulation of T Cell Signaling

The methods described herein in some embodiments employ a population of modified T cells that recognize a cancer associated antigen (CAA), wherein the modified T cells comprise one or more modifications to a population of input T cells that results in expression of a microRNA (miRNA) having the same seed sequence as miR-181a or increases the expression and/or activity of an endogenous miR-181a. In some embodiments, the modified T cells comprise an exogenous nucleic acid molecule encoding the miRNA. In some embodiments, the miRNA is miR-181a. In some embodiments, the modified T cells comprise one or more modifications that increase the expression of endogenous miR-181a-1 and/or miR-181a-2. The modified T cells have a lower T cell receptor (TCR) signaling threshold and/or increased TCR sensitivity to the CAA compared to the input T cells. In some embodiments, the modified T cells recognize one or more additional CAAs. In some embodiments, the input T cells are polyclonal. In some embodiments, the input T cells are isolated from a solid tumor, and the CAA and/or the one or more additional CAAs are expressed by or associated with cancer cells from the solid tumor. In some embodiments, the input T cells are isolated from blood, and the CAA and/or the one or more additional CAAs are expressed by or associated with cancer cells from a hematological malignancy. In some embodiments, the modified T cells have reduced susceptibility to immune suppression and/or exhaustion compared to the input T cells. In some embodiments, the modified T cells comprise an additional modification that further reduces their susceptibility to immune suppression and/or exhaustion.

### Erogenous Nucleic Acid Molecule

The methods described herein in some embodiments employ a population of modified T cells that recognize a CAA, wherein the modified T cells comprise an exogenous nucleic acid molecule encoding an miRNA having the same seed sequence as miR-181a. In some embodiments, the miRNA seed sequence comprises or consists of the nucleotide sequence of SEQ ID NO: 1. In some embodiments, the miRNA is miR-181a. In some embodiments, the miRNA comprises or consists of the nucleotide sequence of SEQ ID NO: 2. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the exogenous nucleic acid encodes a precursor miRNA (pre-miRNA) comprising the miRNA. In some embodiments, the exogenous nucleic acid encodes a primary miRNA (pri-miRNA) comprising the miRNA. In some embodiments, the pre-miRNA or pri-miRNA comprises a loop region having the nucleotide sequence of SEQ ID NO: 4 or 5, or a variant thereof comprising up to 3 nucleotide substitutions. In some embodiments, the pre-miRNA or pri-miRNA comprises a loop region having the nucleotide sequence of SEQ ID NO: 4 or 5. In some embodiments, the pre-miRNA or pri-miRNA comprises a stem having the nucleotide sequences of SEQ ID NOs: 2 and 3, or a variant thereof comprising up to 3 nucleotide substitutions. In some embodiments, the pre-miRNA or pri-miRNA comprises a stem having the nucleotide sequences of SEQ ID NOs: 2 and 3. In some embodiments, the pre-miRNA is the miR-181a-1 or miR-181a-2 pre-miRNA. In some embodiments, the pre-miRNA comprises or consists of the nucleotide sequence of SEQ ID NO: 6 or 7. In some embodiments, the pri-miRNA is the miR-181a-1 or miR-181a-2 pri-miRNA. In some embodiments, the pri-miRNA comprises or consists of the nucleotide sequence of SEQ ID NO: 8 or 9. In some embodiments, the exogenous nucleic acid molecule comprises a regulatory element operably linked to the sequence encoding the miRNA. In some embodiments, the regulatory element is the miR-181a-1 or miR-181a-2 promoter. In some embodiments, the regulatory element is a constitutively active promoter. In some embodiments, the constitutively active promoter is selected from SV40, CMV, UBC, EF1A, PGK or CAGG. In some embodiments, the regulatory element is a conditional promoter, enhancer, or transactivator. In some embodiments, the conditional promoter, enhancer, or transactivator is an inducible promoter, enhancer, or transactivator or a repressible promoter, enhancer, or transactivator. In some embodiments, the promoter comprises a Lac operator sequence, a tetracycline operator sequence, a galactose operator sequence or a doxycycline operator sequence, or is an analog thereof. The modified T cells have a lower T cell receptor (TCR) signaling threshold and/or increased TCR sensitivity to the CAA compared to input T cells lacking the exogenous nucleic acid molecule from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise an exogenous nucleic acid molecule encoding an miRNA comprising (such as consisting of) the sequence of miR-181a. In some embodiments, the miRNA comprises or consists of the nucleotide sequence of SEQ ID NO: 2. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the exogenous nucleic acid encodes a precursor miRNA (pre-miRNA) comprising the miRNA. In some embodiments, the exogenous nucleic acid encodes a primary miRNA (pri-miRNA) comprising the miRNA. In some embodiments, the pre-miRNA or pri-miRNA comprises a loop region having the nucleotide sequence of SEQ ID NO: 4 or 5, or a variant thereof comprising up to 3 nucleotide substitutions. In some embodiments, the pre-miRNA or pri-miRNA comprises a loop region having the nucleotide sequence of SEQ ID NO: 4 or 5. In some embodiments, the pre-miRNA or pri-miRNA comprises a stem having the nucleotide sequences of SEQ ID NOs: 2 and 3, or a variant thereof comprising up to 3 nucleotide substitutions. In some embodiments, the pre-miRNA or pri-miRNA comprises a stem having the nucleotide sequences of SEQ ID NOs: 2 and 3. In some embodiments, the pre-miRNA is the miR-181a-1 or miR-181a-2 pre-miRNA. In some embodiments, the pre-miRNA comprises or consists of the nucleotide sequence of SEQ ID NO: 6 or 7. In some embodiments, the pri-miRNA is the miR-181a-1 or miR-181a-2 pri-miRNA. In some embodiments, the pri-miRNA comprises or consists of the nucleotide sequence of SEQ ID NO: 8 or 9. The modified T cells have a lower T cell receptor (TCR) signaling threshold and/or increased TCR sensitivity to the CAA compared to input T cells lacking the exogenous nucleic acid molecule from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise an exogenous nucleic acid molecule encoding a pre-miRNA comprising an miRNA having the same seed sequence as miR-181a. In some embodiments, the miRNA seed sequence comprises or consists of the nucleotide sequence of SEQ ID NO: 1. In some embodiments, the miRNA is miR-181a. In some embodiments, the miRNA comprises or consists of the nucleotide sequence of SEQ ID NO: 2. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the pre-miRNA comprises a loop region having the nucleotide sequence of SEQ ID NO: 4 or 5, or a variant thereof comprising up to 3 nucleotide substitutions. In some embodiments, the pre-miRNA comprises a loop region having the nucleotide sequence of SEQ ID NO: 4 or 5. In some embodiments, the pre-miRNA comprises a stem having the nucleotide sequences of SEQ ID NOs: 2 and 3, or a variant thereof comprising up to 3 nucleotide substitutions. In some embodiments, the pre-miRNA comprises a stem having the nucleotide sequences of SEQ ID NOs: 2 and 3. In some embodiments, the pre-miRNA is the miR-181a-1 or miR-181a-2 pre-miRNA. In some embodiments, the pre-miRNA comprises or consists of the nucleotide sequence of SEQ ID NO: 6 or 7. The modified T cells have a lower T cell receptor (TCR) signaling threshold and/or increased TCR sensitivity to the CAA compared to input T cells lacking the exogenous nucleic acid molecule from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise an exogenous nucleic acid molecule encoding a pre-miRNA comprising an miRNA comprising (such as consisting of) the sequence of miR-181a. In some embodiments, the miRNA comprises or consists of the nucleotide sequence of SEQ ID NO: 2. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the pre-miRNA comprises a loop region having the nucleotide sequence of SEQ ID NO: 4 or 5, or a variant thereof comprising up to 3 nucleotide substitutions. In some embodiments, the pre-miRNA comprises a loop region having the nucleotide sequence of SEQ ID NO: 4 or 5. In some embodiments, the pre-miRNA comprises a stem having the nucleotide sequences of SEQ ID NOs: 2 and 3, or a variant thereof comprising up to 3 nucleotide substitutions. In some embodiments, the pre-miRNA comprises a stem having the nucleotide sequences of SEQ ID NOs: 2 and 3. In some embodiments, the pre-miRNA is the miR-181a-1 or miR-181a-2 pre-miRNA. In some embodiments, the pre-miRNA comprises or consists of the nucleotide sequence of SEQ ID NO: 6 or 7. The modified T cells have a lower T cell receptor (TCR) signaling threshold and/or increased TCR sensitivity to the CAA compared to input T cells lacking the exogenous nucleic acid molecule from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise an exogenous nucleic acid molecule encoding a pri-miRNA comprising an miRNA having the same seed sequence as miR-181a. In some embodiments, the miRNA seed sequence comprises or consists of the nucleotide sequence of SEQ ID NO: 1. In some embodiments, the miRNA is miR-181a. In some embodiments, the miRNA comprises or consists of the nucleotide sequence of SEQ ID NO: 2. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the pri-miRNA comprises a loop region having the nucleotide sequence of SEQ ID NO: 4 or 5, or a variant thereof comprising up to 3 nucleotide substitutions. In some embodiments, the pri-miRNA comprises a loop region having the nucleotide sequence of SEQ ID NO: 4 or 5. In some embodiments, the pri-miRNA comprises a stem having the nucleotide sequences of SEQ ID NOs: 2 and 3, or a variant thereof comprising up to 3 nucleotide substitutions. In some embodiments, the pri-miRNA comprises a stem having the nucleotide sequences of SEQ ID NOs: 2 and 3. In some embodiments, the pri-miRNA is the miR-181a-1 or miR-181a-2 pri-miRNA. In some embodiments, the pri-miRNA comprises or consists of the nucleotide sequence of SEQ ID NO: 8 or 9. The modified T cells have a lower T cell receptor (TCR) signaling threshold and/or increased TCR sensitivity to the CAA compared to input T cells lacking the exogenous nucleic acid molecule from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise an exogenous nucleic acid molecule encoding a pri-miRNA comprising an miRNA comprising (such as consisting of) the sequence of miR-181a. In some embodiments, the miRNA comprises or consists of the nucleotide sequence of SEQ ID NO: 2. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the pri-miRNA comprises a loop region having the nucleotide sequence of SEQ ID NO: 4 or 5, or a variant thereof comprising up to 3 nucleotide substitutions. In some embodiments, the pri-miRNA comprises a loop region having the nucleotide sequence of SEQ ID NO: 4 or 5. In some embodiments, the pri-miRNA comprises a stem having the nucleotide sequences of SEQ ID NOs: 2 and 3, or a variant thereof comprising up to 3 nucleotide substitutions. In some embodiments, the pri-miRNA comprises a stem having the nucleotide sequences of SEQ ID NOs: 2 and 3. In some embodiments, the pri-miRNA is the miR-181a-1 or miR-181a-2 pri-miRNA. In some embodiments, the pri-miRNA comprises or consists of the nucleotide sequence of SEQ ID NO: 8 or 9. The modified T cells have a lower T cell receptor (TCR) signaling threshold and/or increased TCR sensitivity to the CAA compared to input T cells lacking the exogenous nucleic acid molecule from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

Modulation of Endogenous miR-181a

The methods described herein in some embodiments employ a population of modified T cells that recognize a CAA, wherein the modified T cells comprise a modification that increases the expression of endogenous miR-181a. In some embodiments, the modification increases the expression of endogenous miR-181a-1. In some embodiments, the modification increases the expression of endogenous miR-181a-2. In some embodiments, the modified T cells comprise a nucleic acid molecule inserted into their genome that upregulates an miR-181a gene (i.e., miR-181a-1 or miR-181a-2). In some embodiments, the modified T cells comprise a modification to the miR-181a gene that leads to an increase in the stability of its mRNA transcript. In some embodiments, the modified T cells comprise a sequence-guided complex that leads to an increase in transcription of the miR-181a gene. The modified T cells have a lower T cell receptor (TCR) signaling threshold and/or increased TCR sensitivity to the CAA compared to input T cells lacking the modification from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise a nucleic acid molecule inserted into their genome that comprises all or a portion of an endogenous miR-181a gene, including a sequence encoding miR-181a. In some embodiments, the endogenous miR-181a gene is miR-181a-1. In some embodiments, the nucleic acid molecule comprises all or a portion of the nucleotide sequence of SEQ ID NO: 8. In some embodiments, the endogenous miR-181a gene is miR-181a-2. In some embodiments, the nucleic acid molecule comprises all or a portion of the nucleotide sequence of SEQ ID NO: 9. In some embodiments, the inserted nucleic acid molecule comprises a regulatory element operably linked to the miR-181a sequence. In some embodiments, the regulatory element is that which endogenously drives expression of the miR-181a sequence, such as the miR-181a-1 or miR-181a-2 promoter. In some embodiments, the regulatory element is a constitutively active promoter. In some embodiments, the constitutively active promoter is selected from SV40, CMV, UBC, EF1A, PGK or CAGG. In some embodiments, the regulatory element is a conditional promoter, enhancer, or transactivator. In some embodiments, the conditional promoter, enhancer, or transactivator is an inducible promoter, enhancer, or transactivator or a repressible promoter, enhancer, or transactivator. In some embodiments, the promoter comprises a Lac operator sequence, a tetracycline operator sequence, a galactose operator sequence or a doxycycline operator sequence, or is an analog thereof. The modified T cells have a lower T cell receptor (TCR) signaling threshold and/or increased TCR sensitivity to the CAA compared to input T cells lacking the inserted nucleic acid molecule from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise a nucleic acid molecule inserted into their genome that upregulates endogenous miR-181a-1 or miR-181a-2. In some embodiments, endogenous miR-181a-1 is upregulated. In some embodiments, endogenous miR-181a-2 is upregulated. In some embodiments, the inserted nucleic acid molecule comprises a regulatory element capable of increasing transcription of the miR-181a gene. In some embodiments, the regulatory element is capable of recruiting the RNA polymerase II machinery. In some embodiments, the regulatory element is a promoter, and replaces the endogenous promoter of the miR-181a gene. In some embodiments, the promoter is a constitutively active promoter. In some embodiments, the constitutively active promoter is selected from SV40, CMV, UBC, EF1A, PGK or CAGG. In some embodiments, the regulatory element is a conditional promoter, enhancer, or transactivator. In some embodiments, the conditional promoter, enhancer, or transactivator is an inducible promoter, enhancer, or transactivator or a repressible promoter, enhancer, or transactivator. In some embodiments, the promoter comprises a Lac operator sequence, a tetracycline operator sequence, a galactose operator sequence or a doxycycline operator sequence, or is an analog thereof. In some embodiments, the modified T cells comprise nucleic acid molecules inserted into their genome such that both endogenous miR-181 genes are upregulated. The modified T cells have a lower T cell receptor (TCR) signaling threshold and/or increased TCR sensitivity to the CAA compared to input T cells lacking the inserted nucleic acid molecule from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise a modification to the endogenous miR-181a-1 or miR-181a-2 gene that leads to an increase in the stability of its mRNA transcript. In some embodiments, the endogenous miR-181a-1 gene is modified to increase the stability of its mRNA transcript. In some embodiments, the endogenous miR-181a-2 gene is modified to increase the stability of its mRNA transcript. In some embodiments, both endogenous miR-181a genes are modified to increase the stability of their RNA transcript. The modified T cells have a lower T cell receptor (TCR) signaling threshold and/or increased TCR sensitivity to the CAA compared to input T cells lacking the modification from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

In some embodiments, the population of modified T cells recognize a CAA, wherein the modified T cells comprise a sequence-guided complex that leads to an increase in transcription of the endogenous miR-181a-1 or miR-181a-2 gene. In some embodiments, the sequence-guided complex increases the expression of endogenous miR-181a-1. In some embodiments, the sequence-guided complex increases the expression of endogenous miR-181a-2. In some embodiments, the sequence-guided complex comprises a guide nucleotide associated with a fusion protein comprising a) a first domain capable of being targeted to a recognition site complementary to the guide polynucleotide; and b) a second domain that is an activator of RNA polymerase II. In some embodiments, the modified T cells comprise a first nucleic acid molecule that encodes the guide nucleotide. In some embodiments, the first nucleic acid molecule is inserted into the genome of the modified T cells. In some embodiments, the modified T cells comprise a second nucleic acid molecule that encodes the fusion protein. In some embodiments, the second nucleic acid molecule is inserted into the genome of the modified T cells. In some embodiments, the guide nucleotide is an RNA molecule, and the first domain of the fusion protein comprises an RNA-guided endonuclease or variant thereof, such as a nuclease-deficient variant. In some embodiments, the first domain of the fusion protein comprises a nuclease dead CRISPR-associated endonuclease, such as nuclease dead Cas9 (dCas9). In some embodiments, the guide nucleotide is a DNA molecule, and the first domain of the fusion protein comprises an DNA-guided endonuclease or variant thereof, such as a nuclease-deficient variant. In some embodiments, the first domain of the fusion protein comprises a nuclease dead Natronobacterium gregoryi Argonaute (NgAgo) endonuclease. The modified T cells have a lower T cell receptor (TCR) signaling threshold and/or increased TCR sensitivity to the CAA compared to input T cells lacking the modification from which the modified T cells are derived. In some embodiments, the modified T cells have reduced susceptibility to immune suppression and/or exhaustion compared to the input T cells. In some embodiments, the modified T cells have reduced expression of the immune checkpoint inhibitor PD-1. In some embodiments, the modified T cells recognize one or more additional CAAs. In some embodiments, the input T cells are isolated from a solid tumor. In some embodiments, the CAA and/or the one or more additional CAAs are expressed by or associated with cancer cells from the solid tumor.

### Preparation of Modified T Cells

The methods described herein in some embodiments employ methods for the preparation and culture of the modified T cells provided herein.

### Cell Source

The input cells and compositions containing the input cells for engineering typically are isolated from a sample, such as a biological sample, e.g., one obtained from or derived from an individual. In some embodiments, the individual from which the cell is isolated is one having a particular disease or condition or in need of a cell therapy or to which cell therapy will be administered. The individual in some embodiments is a mammal, such as a human, such as an individual in need of a particular therapeutic intervention, such as the adoptive cell therapy for which cells are being isolated, processed, and/or engineered.

Accordingly, the input cells in some embodiments are primary cells, e.g., primary human cells. The samples include tissue, fluid, and other samples taken directly from the individual, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, tumor fragments, body fluids, such as blood, plasma, and serum, tonsils, and bone marrow, including processed samples derived therefrom (such as digested tumor cell suspensions). In some embodiments, the sample from which the cells are derived or isolated is one or more tumor fragments. In some aspects, the sample from which the cells are derived or isolated is blood or a blood-derived sample, or is or is derived from an apheresis or leukapheresis product. Exemplary samples include tumor fragments, whole blood, peripheral blood mononuclear cells (PBMCs), leukocytes, and bone marrow, and/or cells derived therefrom. Samples include, in the context of cell therapy, e.g., adoptive cell therapy, samples from autologous and allogeneic sources.

In some embodiments, the input cells are derived from cell lines, e.g., T cell lines. The cells in some embodiments are obtained from a xenogeneic source, for example, from mouse, rat, non-human primate, and pig.

### Cell Processing, Preparation, and Separation

In some embodiments, isolation of the input cells includes one or more preparation and/or cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as affinity, density, adherent properties, size, sensitivity and/or resistance to particular components.

In some embodiments, cells from one or more tumor fragments from an individual are obtained. The samples, in some aspects, contain tumor-infiltrating lymphocytes (TILs), including T cells. Expansion of TILs can be achieved by culturing one or more tumor fragments, or cells derived therefrom, in the presence of one or more growth promoting substances. See, for example, WO2015157636, WO2016096903, and Wu, R, et al. (2012). Cancer journal (Sudbury, Mass.), 18(2), 160.

In some embodiments, autologous TILs are obtained from the stroma of resected tumors. In some embodiments, tumor samples are obtained from individuals and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase).

Whether prior to or after modification of input T cells to produce the modified T cells described herein, the T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, T cells are expanded by contact with a surface having attached thereto an agent that stimulates a CD3/TCR complex associated signal and optionally a ligand that stimulates a co-stimulatory molecule on the surface of the T cells. In particular, T cell populations may be stimulated, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4⁺ T cells or CD8⁺ T cells, an anti-CD3 antibody and an anti-CD28 antibody can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999). Examples of an anti-CD28 antibody include 9.3, B-T3, XR-CD28 (Diaclone, Besangon, France).

In some embodiments, expansion of lymphocytes, including tumor-infiltrating lymphocytes, such as T cells, is accomplished by any of a number of methods known in the art. For example, in some embodiments, T cells are rapidly expanded using non-specific T cell receptor stimulation in the presence of feeder lymphocytes and interleukin-2 (IL-2), IL-7, IL-15, IL-21, or combinations thereof. In some embodiments, the non-specific T cell receptor stimulus can include, for example, a mouse monoclonal anti-CD3 antibody (such as OKT3). In other embodiments, T cells are rapidly expanded by stimulation in vitro with one or more antigens recognized by the T cells (including antigenic portions thereof and cells presenting such antigens) in the presence of a T cell growth factor. In some embodiments, the in vitro-induced T cells are rapidly expanded by re-stimulation with the same antigen(s) of the cancer pulsed onto antigen-presenting cells. In some embodiments, the T cells are re-stimulated with irradiated, autologous lymphocytes or with irradiated HLA-A2+ allogeneic lymphocytes and IL-2, for example. Specific tumor reactivity of the expanded TILs can be tested by any method known in the art, e.g., by measuring cytokine release (e.g., interferon-gamma) following co-culture with tumor cells.

In some embodiments, the method comprises enriching cultured TILs for CD3⁺, CD28⁺, CD4⁺, CD8⁺, CD45RA⁺, and/or CD45RO⁺ T cell subpopulations prior to rapid expansion of the cells. In some embodiments, a specific subpopulation of T cells is isolated by positive or negative selection techniques. For example, in some embodiments, T cells are isolated by incubation with anti-CD3/anti-CD28 (i.e., 3×28)-conjugated beads, such as DYNABEADS® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least one, 2, 3, 4, 5, or 6 hours. In some embodiments, the time period is 10 to 24 hours. In some embodiments, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such as in isolating tumor-infiltrating lymphocytes (TIL) from tumor tissue or from immune-compromised individuals. Further, use of longer incubation times can increase the efficiency of capture of CD8T cells. Thus, by simply shortening or lengthening the time T cells are allowed to bind to the CD3/CD28 beads and/or by increasing or decreasing the ratio of beads to T cells, subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In some embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD 16, HLA-DR, and CD8. In some embodiments, it may be desirable to enrich for or positively select for regulatory T cells which typically express CD4⁺, CD25⁺, CD62Lhi, GITR⁺, and FoxP3⁺. Alternatively, in some embodiments, T regulatory cells are depleted by anti-CD25 conjugated beads or other similar methods of selection.

For isolation of a desired population of cells by positive or negative selection, the concentration of cells and surface (e.g., particles such as beads) can be varied. In some embodiments, it may be desirable to significantly decrease the volume in which beads and cells are mixed together (i.e., increase the concentration of cells), to ensure maximum contact of cells and beads. For example, in some embodiments, a concentration of about 2 billion cells/ml is used. In some embodiments, a concentration of about 1 billion cells/ml is used. In some embodiments, greater than about 100 million cells/ml is used. In some embodiments, a concentration of cells of about any of 10, 15, 20, 25, 30, 35, 40, 45, or 50 million cells/ml is used. In some embodiments, a concentration of cells of about any of 75, 80, 85, 90, 95, or 100 million cells/ml is used. In some embodiments, a concentration of about 125 or about 150 million cells/ml is used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8⁺ T cells that normally have weaker CD28 expression.

In some embodiments, following culture of the TILs in IL-2, the T cells are depleted of CD4+ cells and enriched for CD8+ cells using, for example, a CD8 microbead separation (e.g., using a CliniMACS® CD8 microbead system (Miltenyi Biotec)).

In some embodiments, TILs are expanded ex vivo in two stages. In some embodiments, the first stage involves an initial expansion of TILs from one or more tumor fragments or digested tumor cell suspensions, for example, over a 5-week period of time in culture medium with IL-2. In some embodiments, medium exchanges, for example, with fresh IL-2, are performed regularly to ensure continued T cell division and survival during this time. This first stage yields a product (“pre-REP” TIL) that is then used to generate a final TIL infusion product following a “rapid expansion protocol” (REP). In some embodiments, the pre-REP TILs are cryopreserved at this stage in the process for later secondary expansion in the REP. In some embodiments, the pre-REP TILs are used immediately. In some embodiments, the REP involves activating the TILs, for example, through the CD3 complex using an anti-CD3 mAb, optionally in the presence of, for example, a 200:1 ratio of irradiated (5,000 cGy) PBMC feeder cells obtained from the patient (autologous feeders), or from normal healthy donors (allogeneic feeders). In some embodiments, IL-2, such as 6,000 U/ml L-2 (final concentration), is added to drive rapid cell division in the activated TILs after initiation of the REP, for example, at two days following initiation of the REP. In some embodiments, the TILs are then expanded, for example, for another 12 days and diluted as needed, for example, with 1:1 culture medium containing IL-2.

In some examples, cells from the circulating blood of an individual are obtained, e.g., by apheresis or leukapheresis. The samples, in some aspects, contain lymphocytes, including T cells.

In some embodiments, the blood cells collected from the individual are washed, e.g., to remove the plasma fraction and to place the cells in an appropriate buffer or media for subsequent processing steps. In some embodiments, the cells are washed with phosphate buffered saline (PBS).

In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca++/Mg++ free PBS. In certain embodiments, components of a blood cell sample are removed and the cells directly resuspended in culture media.

In some embodiments, the methods include density-based cell separation methods, such as the preparation of white blood cells from peripheral blood by lysing the red blood cells and centrifugation through a Percoll or Ficoll gradient.

### Cryopreservation

In some embodiments, the preparation methods include steps for freezing, e.g., cryopreserving, the cells, either before or after isolation, incubation, and/or engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are then frozen to −80° C. at a rate of 1° per minute and stored in the vapor phase of a liquid nitrogen storage tank.

In some embodiments, the provided methods include cultivation, incubation, culture, and/or genetic engineering steps. For example, in some embodiments, provided are methods for incubating and/or engineering the depleted cell populations and culture-initiating compositions.

Thus, in some embodiments, the cell populations are incubated in a culture-initiating composition. The incubation and/or engineering may be carried out in a culture vessel, such as a unit, chamber, well, column, tube, tubing set, valve, vial, culture dish, bag, or other container for culture or cultivating cells.

### Incubation and Culture

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a genetically engineered exogenous protein and/or receptor.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions include temperature suitable for the growth of human T lymphocytes, for example, at least about 25 degrees Celsius, generally at least about 30 degrees, and generally at or about 37 degrees Celsius.

In some aspects, the methods include assessing expression of one or more markers on the surface of the modified T cells or cells being engineered. In one embodiment, the methods include assessing surface expression of one or more surface markers of a particular T cell lineage, for example, by affinity-based detection methods such as by flow cytometry.

### Modification

The methods described herein in some embodiments employ a method of producing a population of modified T cells, comprising introducing into a population of input T cells recognizing a cancer-associated antigen an exogenous nucleic acid molecule encoding an RNA transcript comprising an miRNA, wherein the miRNA comprises a seed sequence having the nucleotide sequence of SEQ ID NO: 1, and wherein the modified T cells have a lower T cell receptor (TCR) signaling threshold and/or increased TCR sensitivity to the cancer-associated antigen as compared to T cells not comprising the exogenous miRNA. In some embodiments, the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6. In some embodiments, the miRNA targets each of the mRNAs encoding PTPN11, PTPN22, DUSP5, and DUSP6. In some embodiments, the RNA transcript comprises a loop region having the nucleotide sequence of SEQ ID NO: 4, or a variant thereof comprising up to 3 nucleotide substitutions. In some embodiments, the RNA transcript comprises a loop region having the nucleotide sequence of SEQ ID NO: 4. In some embodiments, the RNA transcript comprises a stem region having the nucleotide sequence of SEQ ID NO: 3, or a variant thereof comprising up to 3 nucleotide substitutions. In some embodiments, the RNA transcript comprises a stem region having the nucleotide sequence of SEQ ID NO: 3. In some embodiments, the RNA transcript comprises the sequence of SEQ ID NO: 2. In some embodiments, the RNA transcript comprises a sequence corresponding to a precursor miRNA (pre-miRNA). In some embodiments, the sequence corresponding to the pre-miRNA has the nucleotide sequence of SEQ ID NO: 6 or 7. In some embodiments, the RNA transcript comprises a sequence corresponding to a primary miRNA (pri-miRNA). In some embodiments, the sequence corresponding to the pri-miRNA has the nucleotide sequence of SEQ ID NO: 8 or 9. In some embodiments, the exogenous nucleic acid molecule is introduced by viral transduction, transposition, electroporation, or chemical transfection. In some embodiments, the exogenous nucleic acid molecule is introduced into a targeted locus in the genome of the population of input T cells, such as by homologous recombination. In some embodiments, the exogenous nucleic acid molecule is introduced into a random locus in the genome of the population of input T cells. In some embodiments, the method further comprises isolating T cells from an individual, thereby generating the population of input T cells. In some embodiments, the T cells are isolated from a solid tumor in the individual.

Methods of cell modification, such as introducing nucleic acid molecules into cell genomes, repressing gene expression, and activating gene expression are well known in the art, and described in more detail below.

In some embodiments, the method of producing a population of modified T cells comprises introducing into a population of input T cells recognizing a cancer-associated antigen a first modification that increases the expression of endogenous miR-181a, wherein the modified T cells have a lower TCR signaling threshold and/or increased TCR sensitivity to the cancer-associated antigen as compared to T cells not comprising the exogenous miRNA. In some embodiments, the first modification comprises introducing a nucleic acid molecule encoding a) a fusion protein comprising a nuclease-deficient sequence-guided endonuclease (SGEN) fused to an activator of RNA polymerase II; and b) a guide nucleotide directing the fusion protein to the promoter region of a miR-181a gene. In some embodiments, the miR-181a gene is miR-181a-1. In some embodiments, the miR-181a gene is miR-181a-2. In some embodiments, the first modification comprises introducing a nucleic acid molecule that increases miR-181a expression into the genome of the T cells. In some embodiments, the nucleic acid molecule encodes miR-181a. In some embodiments, the nucleic acid molecule comprises a promoter sequence and is inserted such that the promoter sequence is operably linked to an miR-181a gene (miR-181a-1 or miR-181a-2). In some embodiments, the nucleic acid molecule is introduced by viral transduction, transposition, electroporation, or chemical transfection. In some embodiments, the nucleic acid molecule is introduced into a targeted locus in the genome of the population of input T cells, such as by homologous recombination. In some embodiments, the nucleic acid molecule is introduced into a random locus in the genome of the population of input T cells. In some embodiments, the first modification comprises a modification to an miR-181a gene that leads to an increase in the stability of the mRNA transcript of the miR-181a gene. In some embodiments, the method further comprises isolating T cells from an individual, thereby generating the population of input T cells. In some embodiments, the T cells are isolated from a solid tumor in the individual.

### Compositions

The methods described herein in some embodiments employ compositions containing the engineered cells produced by the provided methods. Among the compositions are pharmaceutical compositions and formulations for administration, such as for adoptive cell therapy.

In some embodiments, the cells and cell populations are administered to an individual in the form of a composition, such as a pharmaceutical composition. In some embodiments, the pharmaceutical composition further comprises other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc. In some embodiments, the cell populations in the composition are in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid.

In some embodiments, the choice of carrier in the pharmaceutical composition is determined in part by the particular modified T cells, as well as by the particular method used to administer the modified T cells. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof may be present in an amount of about 0.0001% to about 2% by weight of the total composition.

In addition, buffering agents in some aspects are included in the composition. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

In certain embodiments, a pharmaceutical composition comprising a cell population described herein can be formulated as an inclusion complex, such as cyclodextrin inclusion complex, or as a liposome. Liposomes can serve to target the host cells (e.g., T cells or NK cells) to a particular tissue. Many methods are available for preparing liposomes, such as those described in, for example, Szoka et al., Ann. Rev. Biophys. Bioeng., 9: 467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known to those of ordinary skill in the art. Such systems can avoid repeated administrations of the composition, thereby increasing convenience to the subject and the physician.

The pharmaceutical composition in some embodiments comprises the cells in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

### Methods of Cell Modification

In some embodiments, a modified T cell described herein comprises one or more exogenous nucleic acid molecules. In some embodiments, expression, activity, and/or function of one or more genes is modified in a modified T cell described herein. Provided are methods for effecting such modifications.

In some embodiments, the modification is introduction of an exogenous nucleic acid molecule. In some embodiments, the exogenous nucleic acid comprises a sequence encoding miR-181a. In some embodiments, the exogenous nucleic acid comprises a sequence encoding pre-miR-181a-1 or pre-miR-181a-2. In some embodiments, the exogenous nucleic acid comprises a sequence encoding pri-miR-181a-1 or pri-miR-181a-2. In some embodiments, the exogenous nucleic acid comprises a sequence encoding a modified miR-181a. In some embodiments, the modified miR-181a comprises the seed sequence of human miR-181a. In some embodiments, the exogenous nucleic acid comprises a regulatory element, such as a promoter. In some embodiments, the exogenous nucleic acid comprises a sequence encoding a T cell activating receptor. In some embodiments, the exogenous nucleic acid comprises a sequence encoding a dominant negative ligand for an immune checkpoint inhibitor.

In some embodiments, the modification is gene repression. In some embodiments, the gene repression is carried out by effecting a disruption in the gene (gene editing), such as a knock-out, insertion, missense or frameshift mutation, such as a biallelic frameshift mutation, deletion of all or part of the gene, e.g., one or more exon or portion thereof, and/or knock-in. Such disruptions in some embodiments are effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of a gene or a portion thereof.

In some embodiments, the gene repression is carried out by introducing an inhibitory nucleic acid molecule targeting the gene. In some embodiments, the inhibitory nucleic acid includes a small interfering RNA (siRNA), a microRNA-adapted shRNA, a short hairpin RNA (shRNA), a hairpin siRNA, a microRNA (miRNA-precursor) or a microRNA (miRNA).

In some embodiments, the modification is gene activation. In some embodiments, the gene activation is carried out by increasing the copy number of the gene, such as knock-in of the gene, or by activating the transcription and/or translation of the gene. Knock-in in some embodiments is effected by RNA-guided nucleases such as a CRISPR-associated nuclease (Cas) in combination with a donor template comprising a coding sequence for the gene. Transcriptional activation in some embodiments is effected by RNA-guided nucleases such as a CRISPR-associated nuclease (Cas) comprising a nuclease-inactivating mutation and fused to a transcriptional activator.

### Introduction of Nucleic Acid Molecules

In some embodiments, a nucleic acid molecule described herein is transferred into cells (such as T cells) using recombinant infectious virus particles, such as, e.g., vectors derived from simian virus 40 (SV40), adenoviruses, or adeno-associated virus (AAV). In some embodiments, the nucleic acids are transferred into cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November; 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, the nucleic acids described herein are transferred into cells (such as T cells) via electroporation (see, e.g., Chicaybam et al., (2013) PLoS ONE 8(3): e60298 and Van Tedeloo et al. (2000) Gene Therapy 7(16): 1431-1437). In some embodiments, the nucleic acids are transferred into cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the genetically engineered nucleic acids encoding the exogenous protein include those described, e.g., in international patent application Publication No.: WO2014055668, and U.S. Pat. No. 7,446,190.

In some embodiments, a nucleic acid described herein is introduced into a random locus in the modified T cell. In some embodiments, the nucleic acid is inserted into the random locus. In some embodiments, the nucleic acid replaces all or a portion of the random locus. Techniques for introduction of a transgene using genetic engineering, such as by viral transduction, are well known in the art. See for example WO9429438, WO9533824, WO9712052, WO200111067, WO200218609, WO2013014537, and WO2014026110.

In some embodiments, a nucleic acid described herein is integrated into a target locus in the modified T cell. In some embodiments, the nucleic acid comprises sequences that allow for integration at the target locus by homologous recombination. In some embodiments, the nucleic acid comprises flanking sequences that are homologous to sequences at the target locus. In some embodiments, the nucleic acid is inserted into the target locus. In some embodiments, the nucleic acid replaces all or a portion of the target locus. In some embodiments, integration into the target locus is mediated by a designer nuclease selected from zinc-finger nucleases (ZFNs), transcription activator-like effector nucleases (TALENs), or RNA-guided nucleases (RGNs). In some embodiments, the RGN is a clustered, regularly interspaced, short palindromic repeats (CRISPR)-associated Cas9 (CRISPR-Cas9) nuclease. Techniques for using CRISPR/Cas9-mediated gene knock-in are known in the art. See for example Auer, T. O. et al. (2014) Genome research 24(1): 142-153; Kimura, Y., et al. (2014) Scientific reports, 4; Aida, T., et al. (2015) Genome biology, 16(1): 1; and Park, A., et al. (2014) PloS one, 9(4): e95101.

In some embodiments, a nucleic acid described herein is integrated into a target locus in the modified T cell, wherein the target locus is an miR-181a gene locus. In some embodiments, the miR-181a gene locus is the miR-181a-1 gene locus. In some embodiments, the miR-181a gene locus is the miR-181a-2 gene locus. In some embodiments, the nucleic acid is integrated in the miR-181a gene locus such that the endogenous miR-181a encoded by the locus cannot be expressed. In some embodiments, the nucleic acid is integrated in the miR-181a gene locus such that the endogenous miR-181a encoded by the locus can still be expressed. In some embodiments, the nucleic acid is inserted in the miR-181a gene locus without replacing any sequences. In some embodiments, the nucleic acid is inserted upstream of the sequence encoding primary miR-181a. In some embodiments, the nucleic acid is operably linked to a promoter and/or enhancer at the miR-181a gene locus. In some other embodiments, the nucleic acid replaces all or a portion of the miR-181a gene locus, such as all or a portion of the sequence encoding primary miR-181a. In some embodiments, the nucleic acid replaces all or most of the miR-181a gene locus, and comprises regulatory sequences sufficient for expression of the product encoded by the nucleic acid (e.g., a modified miR-181a). In some embodiments, the nucleic acid replaces a portion of the miR-181a gene locus, such as a portion of the sequence encoding primary miR-181a. In some embodiments, the nucleic acid does not replace one or more of the regulatory sequences at the miR-181a gene locus, and comprises a sequence encoding a modified miR-181a operably linked to one or more of the remaining regulatory sequences, such that the modified miR-181a is regulated similarly to the endogenous miR-181a prior to integration of the modified miR-181a. In some such embodiments, the nucleic acid may comprise a modified portion of primary miR-181a, such that the modified miR-181a is expressed as a chimera including a portion of the endogenous miR-181a.

Additional nucleic acids for introduction include those comprising (1) genes to improve the efficacy of therapy, such as by promoting viability and/or function of the modified T cells (e.g., genes encoding activating receptors or dominant negative ligands for immune checkpoint inhibitors); (2) genes to provide a genetic marker for selection and/or evaluation of the cells, such as to assess in vivo survival or localization; and/or (3) genes to improve safety, for example, by making the modified T cells susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

### Gene Repression

In some embodiments, the repression of the expression, activity, and/or function of the gene is carried out by disrupting the gene. In some aspects, the gene is disrupted so that its expression is reduced by at least at or about 20, 30, or 40%, generally at least at or about 50, 60, 70, 80, 90, or 95% as compared to the expression in the absence of the gene disruption or in the absence of the components introduced to effect the disruption.

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g. an endonuclease, such as a gene-targeted nuclease. In some aspects, the breaks are induced in the coding region of the gene, e.g. in an exon. For example, in some embodiments, the induction occurs near the N-terminal portion of the coding region, e.g. in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon.

In some aspects, the presence of an insertion, deletion, translocation, frameshift mutation, and/or a premature stop codon results in repression of the expression, activity, and/or function of the gene.

In some embodiments, the repression is transient or reversible, such that expression of the gene is restored at a later time. In other embodiments, the repression is not reversible or transient, e.g., is permanent.

In some embodiments, gene repression is achieved using antisense techniques, such as by RNA interference (RNAi), short interfering RNA (siRNA), short hairpin (shRNA), and/or ribozymes are used to selectively suppress or repress expression of the gene. siRNA technology includes that based on RNAi utilizing a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions.

### DNA-Targeting Molecules and Complexes; Targeted Endonucleases

In some embodiments, the repression is achieved using a DNA-targeting molecule, such as a DNA-binding protein or DNA-binding nucleic acid, or complex, compound, or composition, containing the same, which specifically binds to or hybridizes to the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the DNA-targeting molecule, complex, or combination contains a DNA-binding molecule and one or more additional domain, such as an effector domain to facilitate the repression or disruption of the gene. For example, in some embodiments, the gene disruption is carried out by fusion proteins that comprise DNA-binding proteins and a heterologous regulatory domain or functional fragment thereof. In some aspects, domains include, e.g., transcription factor domains such as activators, repressors, co-activators, co-repressors, silencers, oncogenes, DNA repair enzymes and their associated factors and modifiers, DNA rearrangement enzymes and their associated factors and modifiers, chromatin associated proteins and their modifiers, e.g. kinases, acetylases and deacetylases, and DNA modifying enzymes, e.g. methyltransferases, topoisomerases, helicases, ligases, kinases, phosphatases, polymerases, endonucleases, and their associated factors and modifiers. See, for example, U.S. Patent Application Publication Nos. 20050064474; 20060188987 and 2007/0218528, incorporated by reference in their entireties herein, for details regarding fusions of DNA-binding domains and nuclease cleavage domains. In some aspects, the additional domain is a nuclease domain. Thus, in some embodiments, gene disruption is facilitated by gene or genome editing, using engineered proteins, such as nucleases and nuclease-containing complexes or fusion proteins, composed of sequence-specific DNA-binding domains fused to or complexed with non-specific DNA-cleavage molecules such as nucleases.

In some aspects, these targeted chimeric nucleases or nuclease-containing complexes carry out precise genetic modifications by inducing targeted double-stranded breaks or single-stranded breaks, stimulating the cellular DNA-repair mechanisms, including error-prone non-homologous end joining (NHEJ) and homology-directed repair (HDR). In some embodiments the nuclease is an endonuclease, such as a zinc finger nuclease (ZFN), TALE nuclease (TALEN), an RNA-guided endonuclease (RGEN), such as a CRISPR-associated (Cas) protein, or a meganuclease.

In some embodiments, a donor nucleic acid, e.g., a donor plasmid or nucleic acid encoding the exogenous protein and/or recombinant receptor, is inserted by HDR at the site of gene editing following the introduction of the DSBs. Thus, in some embodiments, the disruption of the gene and the introduction of the nucleic acid encoding the exogenous protein and/or recombinant receptor are carried out simultaneously, whereby the gene is disrupted in part by knock-in or insertion of the nucleic acid encoding the exogenous protein and/or recombinant receptor.

In some embodiments, no donor nucleic acid is inserted. In some aspects, NHEJ-mediated repair following introduction of DSBs results in insertion or deletion mutations that can cause gene disruption, e.g., by creating missense mutations or frameshifts.

### ZFPs and ZFNs; TALs, TALEs, and TALENs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs. See Lloyd et al., Fronteirs in Immunology, 4(221), 1-7 (2013).

In some embodiments, the DNA-targeting molecule comprises one or more zinc-finger proteins (ZFPs) or domains thereof that bind to DNA in a sequence-specific manner. A ZFP or domain thereof is a protein or domain within a larger protein, that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP.

Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

In some aspects, repression of the gene is carried out by contacting a first target site in the gene with a first ZFP, thereby repressing the gene. In some embodiments, the target site in the gene is contacted with a fusion ZFP comprising six fingers and the regulatory domain, thereby inhibiting expression of the gene.

In some embodiments, the step of contacting further comprises contacting a second target site in the gene with a second ZFP. In some aspects, the first and second target sites are adjacent. In some embodiments, the first and second ZFPs are covalently linked. In some aspects, the first ZFP is a fusion protein comprising a regulatory domain or at least two regulatory domains. In some embodiments, the first and second ZFPs are fusion proteins, each comprising a regulatory domain or each comprising at least two regulatory domains. In some embodiments, the regulatory domain is a transcriptional repressor, a transcriptional activator, an endonuclease, a methyl transferase, a histone acetyltransferase, or a histone deacetylase.

In some embodiments, the ZFP is encoded by a ZFP nucleic acid operably linked to a promoter. In some aspects, the method further comprises the step of first administering the nucleic acid to the cell in a lipid:nucleic acid complex or as naked nucleic acid. In some embodiments, the ZFP is encoded by an expression vector comprising a ZFP nucleic acid operably linked to a promoter. In some embodiments, the ZFP is encoded by a nucleic acid operably linked to an inducible promoter. In some aspects, the ZFP is encoded by a nucleic acid operably linked to a weak promoter.

In some embodiments, the target site is upstream of a transcription initiation site of the gene. In some aspects, the target site is adjacent to a transcription initiation site of the gene. In some aspects, the target site is adjacent to an RNA polymerase pause site downstream of a transcription initiation site of the gene.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type IIS restriction enzyme and one or more zinc finger binding domains, which may or may not be engineered. In some embodiments, the cleavage domain is from the Type IIS restriction endonuclease Fok I. Fok I generally catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982.]

In some embodiments, ZFNs target a gene present in the modified T cell. In some aspects, the ZFNs efficiently generate a double strand break (DSB), for example at a predetermined site in the coding region of the gene. Typical regions targeted include exons, regions encoding N-terminal regions, first exon, second exon, and promoter or enhancer regions. In some embodiments, transient expression of the ZFNs promotes highly efficient and permanent disruption of the target gene in the modified T cells. In particular, in some embodiments, delivery of the ZFNs results in the permanent disruption of the gene with efficiencies surpassing 50%.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins. Gaj et al., Trends in Biotechnology, 2013, 31(7), 397-405. In some embodiments, commercially available zinc fingers are used or are custom designed. (See, for example, Sigma-Aldrich catalog numbers CSTZFND, CSTZFN, CTI-1KT, and PZD0020).

### TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein. A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NG binds to T and non-canonical (atypical) RVDs are also known. See, U.S. Patent Publication No. 20110301073. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE-nuclease (TALEN). In some aspects the TALEN is a fusion protein comprising a DNA-binding domain derived from a TALE and a nuclease catalytic domain to cleave a nucleic acid target sequence. In some embodiments, the TALE DNA-binding domain has been engineered to bind a target sequence within genes that encode the target antigen and/or the immunosuppressive molecule. For example, in some aspects, the TALE DNA-binding domain may target CD38 and/or an adenosine receptor, such as A2AR.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation (Critchlow and Jackson, Trends Biochem Sci. 1998 October; 23(10):394-8) or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., Trends in Biotechnology, 2013, 31(7), 397-405). A library of TALENs targeting 18,740 human protein-coding genes has been constructed (Kim et al., Nature Biotechnology. 31, 251-258 (2013)). Custom-designed TALE arrays are commercially available through Cellectis Bioresearch (Paris, France), Transposagen Biopharmaceuticals (Lexington, Ky., USA), and Life Technologies (Grand Island, N.Y., USA). Specifically, TALENs that target CD38 are commercially available (See Gencopoeia, catalog numbers HTN222870-1, HTN222870-2, and HTN222870-3, available on the World Wide Web at www.genecopoeia.com/product/search/detail.php?prt=26&cid=&key=HTN222870). Exemplary molecules are described, e.g., in U.S. Patent Publication Nos. US 2014/0120622, and 2013/0315884.

In some embodiments the TALENs are introduced as transgenes encoded by one or more plasmid vectors. In some aspects, the plasmid vector can contain a selection marker which provides for identification and/or selection of cells which received said vector.

### RGENs (CRISPR/Cas Systems)

In some embodiments, the repression is carried out using one or more DNA-binding nucleic acids, such as disruption via an RNA-guided endonuclease (RGEN), or other form of repression by another RNA-guided effector molecule. For example, in some embodiments, the repression is carried out using clustered regularly interspaced short palindromic repeats (CRISPR) and CRISPR-associated (Cas) proteins. See Sander and Joung, Nature Biotechnology, 32(4): 347-355.

In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

In some embodiments, the CRISPR/Cas nuclease or CRISPR/Cas nuclease system includes a non-coding RNA molecule (guide) RNA, which sequence-specifically binds to DNA, and a Cas protein (e.g., Cas9), with nuclease functionality (e.g., two nuclease domains).

In some embodiments, one or more elements of a CRISPR system is derived from a type I, type H, or type III CRISPR system. In some embodiments, one or more elements of a CRISPR system is derived from a particular organism comprising an endogenous CRISPR system, such as Streptococcus pyogenes.

In some embodiments, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. In some embodiments, the target site is selected based on its location immediately 5′ of a protospacer adjacent motif (PAM) sequence, such as typically NGG, or NAG. In this respect, the gRNA is targeted to the desired sequence by modifying the first 20 nucleotides of the guide RNA to correspond to the target DNA sequence.

In some embodiments, the CRISPR system induces DSBs at the target site, followed by disruptions as discussed herein. In other embodiments, Cas9 variants, deemed “nickases” are used to nick a single strand at the target site. In some aspects, paired nickases are used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

In general, a CRISPR system is characterized by elements that promote the formation of a CRISPR complex at the site of a target sequence. Typically, the In the context of formation of a CRISPR complex, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The target sequence may comprise any polynucleotide, such as DNA or RNA polynucleotides. In some embodiments, the target sequence is located in the nucleus or cytoplasm of the cell. In some embodiments, the target sequence may be within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. In some embodiments, the tracr sequence has sufficient complementarity to a tracr mate sequence to hybridize and participate in formation of the CRISPR complex.

As with the target sequence, in some embodiments, complete complementarity is not necessarily needed. In some embodiments, the tracr sequence has at least 50%, 60%, 70%, 80%, 90%, 95% or 99% of sequence complementarity along the length of the tracr mate sequence when optimally aligned. In some embodiments, one or more vectors driving expression of one or more elements of the CRISPR system are introduced into the cell such that expression of the elements of the CRISPR system direct formation of the CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. In some embodiments, CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g. about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of the CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to the cell.

In some embodiments, a vector comprises a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2. In some embodiments, the unmodified CRISPR enzyme has DNA cleavage activity, such as Cas9. In some embodiments the CRISPR enzyme is Cas9, and may be Cas9 from S. pyogenes or S. pneumoniae. In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50,100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. In some embodiments, one or more codons (e.g. 1, 2, 3, 4, 5, 10, 15, 20, 25, 50, or more, or all codons) in a sequence encoding the CRISPR enzyme correspond to the most frequently used codon for a particular amino acid.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net). In some embodiments, a guide sequence is about or more than about 5, 10, 11, 12,13,14,15,16,17,18,19,20,21,22,23,24,25,26,27,28,29,30,35,40,45,50,75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of the CRISPR complex to a target sequence may be assessed by any suitable assay. For example, the components of the CRISPR system sufficient to form the CRISPR complex, including the guide sequence to be tested, may be provided to the cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of the CRISPR complex, including the guide sequence to be tested and a control guide sequence different from the test guide sequence, and comparing binding or rate of cleavage at the target sequence between the test and control guide sequence reactions.

A guide sequence may be selected to target any target sequence. In some embodiments, the target sequence is a sequence within a genome of a cell. Exemplary target sequences include those that are unique in the target genome. In some embodiments, a guide sequence is selected to reduce the degree of secondary structure within the guide sequence. Secondary structure may be determined by any suitable polynucleotide folding algorithm.

In general, a tracr mate sequence includes any sequence that has sufficient complementarity with a tracr sequence to promote one or more of (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences.

Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In some aspects, loop forming sequences for use in hairpin structures are four nucleotides in length, and have the sequence GAAA. However, longer or shorter loop sequences may be used, as may alternative sequences. In some embodiments, the sequences include a nucleotide triplet (for example, AAA), and an additional nucleotide (for example C or G). Examples of loop forming sequences include CAAA and AAAG. In some embodiments, the transcript or transcribed polynucleotide sequence has at least two or more hairpins. In some embodiments, the transcript has two, three, four or five hairpins. In a further embodiment, the transcript has at most five hairpins. In some embodiments, the single transcript further includes a transcription termination sequence, such as a polyT sequence, for example six T nucleotides.

In some embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains.

Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CR ISPR enzyme are described in US20110059502, incorporated herein by reference. In some embodiments, a tagged CRISPR enzyme is used to identify the location of a target sequence.

In some embodiments, a CRISPR enzyme in combination with (and optionally complexed with) a guide sequence is delivered to the cell.

In some aspects, target polynucleotides are modified in a eukaryotic cell. In some embodiments, the method comprises allowing the CRISPR complex to bind to the target polynucleotide to effect cleavage of said target polynucleotide thereby modifying the target polynucleotide, wherein the CRISPR complex comprises the CRISPR enzyme complexed with a guide sequence hybridized to a target sequence within said target polynucleotide, wherein said guide sequence is linked to a tracr mate sequence which in turn hybridizes to a tracr sequence.

In some aspects, the methods include modifying expression of a polynucleotide in a eukaryotic cell. In some embodiments, the method comprises allowing the CRISPR complex to bind to the polynucleotide such that said binding results in increased or decreased expression of said polynucleotide; wherein the CRISPR complex comprises a CRISPR enzyme complexed with a guide sequence hybridized to a target sequence within said polynucleotide, wherein said guide sequence is linked to a tracr mate sequence which in turn hybridizes to a tracr sequence.

### Delivery of Nucleic Acids Encoding the Gene Disrupting Molecules and Complexes

In some aspects, a nucleic acid encoding the DNA-targeting molecule, complex, or combination, is administered or introduced to the cell. The nucleic acid typically is administered in the form of an expression vector, such as a viral expression vector. In some aspects, the expression vector is a retroviral expression vector, an adenoviral expression vector, a DNA plasmid expression vector, or an AAV expression vector. In some aspects, one or more polynucleotides encoding the disruption molecule or complex, such as the DNA-targeting molecule, is delivered to the cell. In some aspects, the delivery is by delivery of one or more vectors, one or more transcripts thereof, and/or one or proteins transcribed therefrom, is delivered to the cell.

In some embodiments, the polypeptides are synthesized in situ in the cell as a result of the introduction of polynucleotides encoding the polypeptides into the cell. In some aspects, the polypeptides could be produced outside the cell and then introduced thereto. Methods for introducing a polynucleotide construct into animal cells are known and include, as non-limiting examples stable transformation methods wherein the polynucleotide construct is integrated into the genome of the cell, transient transformation methods wherein the polynucleotide construct is not integrated into the genome of the cell, and virus mediated methods. In some embodiments, the polynucleotides may be introduced into the cell by for example, recombinant viral vectors (e.g. retroviruses, adenoviruses), liposome and the like. For example, in some aspects, transient transformation methods include microinjection, electroporation, or particle bombardment. In some embodiments, the polynucleotides may be included in vectors, more particularly plasmids or virus, in view of being expressed in the cells.

In some embodiments, viral and non-viral based gene transfer methods can be used to introduce nucleic acids in mammalian cells or target tissues. Such methods can be used to administer nucleic acids encoding components of a CRISPR, ZFP, ZFN, TALE, and/or TALEN system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Fegner, TIBTECH 11:211-217 (1993); Mitani & Caskey, TIBTECH 11:162-166 (1993); Dillon. TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

In some embodiments, delivery is via the use of RNA or DNA viral based systems for the delivery of nucleic acids. Viral vectors in some aspects may be administered directly to patients (in vivo) or they can be used to treat cells in vitro or ex vivo, and then administered to patients. Viral-based systems in some embodiments include retroviral, lentivirus, adenoviral, adeno-associated and herpes simplex virus vectors for gene transfer.

In some aspects, a reporter gene which includes but is not limited to glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP), may be introduced into the cell to encode a gene product which serves as a marker by which to measure the alteration or modification of expression of the gene product. In a further embodiment, the DNA molecule encoding the gene product may be introduced into the cell via a vector. In some embodiments, the gene product is luciferase. In a further embodiment, the expression of the gene product is decreased.

### Inhibitory Nucleic Acid Molecules

In some embodiments, gene repression is achieved using an inhibitory nucleic acid molecule that is an RNA interfering agent, which can be used to selectively suppress or repress expression of the gene. For example, gene repression can be carried out by RNA interference (RNAi), short interfering RNA (siRNA), short hairpin (shRNA), antisense, and/or ribozymes. In some embodiments, RNA interfering agents also can include other RNA species that can be processed intracellularly to produce shRNAs including, but not limited to, RNA species identical to a naturally occurring miRNA precursor or a designed precursor of an miRNA-like RNA.

In some embodiments, an RNA interfering agent is at least a partly double-stranded RNA having a structure characteristic of molecules that are known in the art to mediate inhibition of gene expression through an RNAi mechanism or an RNA strand comprising at least partially complementary portions that hybridize to one another to form such a structure. When an RNA contains complementary regions that hybridize with each other, the RNA will be said to self-hybridize. In some embodiments, an inhibitory nucleic acid, such as an RNA interfering agent, includes a portion that is substantially complementary to a target gene. In some embodiments, an RNA interfering agent targeted to a transcript can also be considered targeted to the gene that encodes and directs synthesis of the transcript. In some embodiments, a target region can be a region of a target transcript that hybridizes with an antisense strand of an RNA interfering agent. In some embodiments, a target transcript can be any RNA that is a target for inhibition by RNA interference.

In some embodiments, an RNA interfering agent is considered to be “targeted” to a transcript and to the gene that encodes the transcript if (1) the RNAi agent comprises a portion, e.g., a strand, that is at least approximately 80%, approximately 85%, approximately 90%, approximately 91%, approximately 92%, approximately 93%, approximately 94%, approximately 95%, approximately 96%, approximately 97%, approximately 98%, approximately 99%, or approximately 100% complementary to the transcript over a region about 15-29 nucleotides in length, e.g., a region at least approximately 15, approximately 17, approximately 18, or approximately 19 nucleotides in length; and/or (2) the Tm of a duplex formed by a stretch of 15 nucleotides of one strand of the RNAi agent and a 15 nucleotide portion of the transcript, under conditions (excluding temperature) typically found within the cytoplasm or nucleus of mammalian cells is no more than approximately 15° C. lower or no more than approximately 10° C. lower, than the Tm of a duplex that would be formed by the same 15 nucleotides of the RNA interfering agent and its exact complement; and/or (3) the stability of the transcript is reduced in the presence of the RNA interfering agent as compared with its absence.

In some embodiments, an RNA interfering agent optionally includes one or more nucleotide analogs or modifications. One of ordinary skill in the art will recognize that RNAi agents can include ribonucleotides, deoxyribonucleotide, nucleotide analogs, modified nucleotides or backbones, etc. In some embodiments, RNA interfering agents may be modified following transcription. In some embodiments, RNA interfering agents can contain one or more strands that hybridize or self-hybridize to form a structure that includes a duplex portion between about 15-29 nucleotides in length, optionally having one or more mismatched or unpaired nucleotides within the duplex.

In some embodiments, the term “short, interfering RNA” (siRNA) refers to a nucleic acid that includes a double-stranded portion between about 15-29 nucleotides in length and optionally further includes a single-stranded overhang (e.g., 1-6 nucleotides in length) on either or both strands. In some embodiments, the double-stranded portion can be between 17-21 nucleotides in length, e.g., 19 nucleotides in length. In some embodiments, the overhangs are present on the 3′ end of each strand, can be about or approximately 2 to 4 nucleotides long, and can be composed of DNA or nucleotide analogs. An siRNA may be formed from two RNA strands that hybridize together, or may alternatively be generated from a longer double-stranded RNA or from a single RNA strand that includes a self-hybridizing portion, such as a short hairpin RNA. One of ordinary skill in the art will appreciate that one or more mismatches or unpaired nucleotides can be present in the duplex formed by the two siRNA strands. In some embodiments, one strand of an siRNA (the “antisense” or “guide” strand) includes a portion that hybridizes with a target nucleic acid, e.g., an mRNA transcript. In some embodiments, the antisense strand is perfectly complementary to the target over about 15-29 nucleotides, sometimes between 17-21 nucleotides, e.g., 19 nucleotides, meaning that the siRNA hybridizes to the target transcript without a single mismatch over this length. However, one of ordinary skill in the art will appreciate that one or more mismatches or unpaired nucleotides may be present in a duplex formed between the siRNA strand and the target transcript.

In some embodiments, a short hairpin RNA (shRNA) is a nucleic acid molecule comprising at least two complementary portions hybridized or capable of hybridizing to form a duplex structure sufficiently long to mediate RNAi (typically between 15-29 nucleotides in length), and at least one single-stranded portion, typically between approximately 1 and 10 nucleotides in length that forms a loop connecting the ends of the two sequences that form the duplex. In some embodiments, the structure may further comprise an overhang. In some embodiments, the duplex formed by hybridization of self-complementary portions of the shRNA may have similar properties to those of siRNAs and, in some cases, shRNAs can be processed into siRNAs by the conserved cellular RNAi machinery. Thus shRNAs can be precursors of siRNAs and can be similarly capable of inhibiting expression of a target transcript. In some embodiments, an shRNA includes a portion that hybridizes with a target nucleic acid, e.g., an mRNA transcript, and can be perfectly complementary to the target over about 15-29 nucleotides, sometimes between 17-21 nucleotides, e.g., 19 nucleotides. However, one of ordinary skill in the art will appreciate that one or more mismatches or unpaired nucleotides may be present in a duplex formed between the shRNA strand and the target transcript.

### Gene Activation

In some embodiments, the enhancement of the expression, activity, and/or function of the gene is carried out by modifying the expression of the endogenous gene, by introducing an exogenous copy of the gene, or by stabilizing and/or de-repressing the gene product. In some aspects, the expression and/or activity of gene is increased by at least or by about 20, 30, or 40%, generally at least or about 50, 60, 70, 80, 90, or 95% as compared to the expression and/or activity in the absence of the gene activation or in the absence of the components introduced to effect the enhancement.

In some embodiments, the expression of the endogenous gene is modified by disrupting a negative regulatory element associated with the gene or a negative transcriptional regulator of the gene, such as by any of the methods of targeted disruption described herein. In some embodiments, the expression of the endogenous gene is modified by introducing a positive regulatory element in association with the gene or a positive transcriptional activator of the gene. Methods for introducing genetic modifications and expressing exogenous proteins are well known in the art.

In some embodiments, the activation is transient or reversible, such that expression of the gene is reduced to unmodified levels at a later time. In other embodiments, the activation is not reversible or transient, e.g., is permanent.

In some embodiments, gene activation is achieved using antisense techniques, such as by RNA interference (RNAi), short interfering RNA (siRNA), short hairpin (shRNA), and/or ribozymes used to selectively suppress or repress expression of negative regulators of the gene.

### DNA-Targeting Molecules and Complexes; Targeted Endonucleases

In some embodiments, the activation is achieved using a DNA-targeting molecule, such as a DNA-binding protein or DNA-binding nucleic acid, or complex, compound, or composition, containing the same, which specifically binds to or hybridizes to a regulatory element associated with the gene. In some embodiments, the DNA-targeting molecule comprises a DNA-binding domain, e.g., a zinc finger protein (ZFP) DNA-binding domain, a transcription activator-like protein (TAL) or TAL effector (TALE) DNA-binding domain, a clustered regularly interspaced short palindromic repeats (CRISPR) DNA-binding domain, or a DNA-binding domain from a meganuclease.

In some embodiments, the DNA-targeting molecule, complex, or combination contains a DNA-binding molecule and one or more additional domain, such as an effector domain to facilitate the activation of the gene. For example, in some embodiments, the gene activation is carried out by fusion proteins that comprise DNA-binding proteins and a heterologous regulatory domain or functional fragment thereof. In some aspects, domains include, e.g., transcription factor domains such as activators, co-activators, oncogenes, DNA repair enzymes and their associated factors and modifiers, DNA rearrangement enzymes and their associated factors and modifiers, chromatin associated proteins and their modifiers, e.g. kinases, acetylases and deacetylases, and DNA modifying enzymes, e.g. methyltransferases, topoisomerases, helicases, ligases, kinases, phosphatases, polymerases, endonucleases, and their associated factors and modifiers.

### RGENs (CRISPR/Cas Systems)

In some embodiments, the activation is carried out using one or more DNA-binding nucleic acids, such as activation via an RNA-guided endonuclease (RGEN), or other form of activation by another RNA-guided effector molecule. For example, in some embodiments, the activation is carried out using CRISPR-associated (Cas) proteins. See Perez-Pinera, P., et al. (2013) Nature methods, 10(10): 973-976.

Both RuvC- and HNH-nuclease domains can be rendered inactive by point mutations (D10A and H840A in SpCas9), resulting in a nuclease dead Cas9 (dCas9) molecule that cannot cleave target DNA. The dCas9 molecule retains the ability to bind to target DNA based on the gRNA targeting sequence. dCas9 can be tagged with transcriptional activators, and targeting these dCas9 fusion proteins to the promoter region results in robust transcriptional activation of downstream target genes. The simplest dCas9-based activators and repressors consist of dCas9 fused directly to a single transcriptional activator, (e.g. VP64). Additionally, more elaborate activation strategies have been developed which result in greater activation of target genes in mammalian cells. These include: co-expression of epitope-tagged dCas9 and antibody-activator effector proteins (e.g. SunTag system), dCas9 fused to several different activation domains in series (e.g. dCas9-VPR) or co-expression of dCas9-VP64 with a “modified scaffold” gRNA and additional RNA-binding “helper activators” (e.g. SAM activators). Importantly, dCas9-mediated gene activation is reversible, since it does not permanently modify the genomic DNA.

A method of treating cancer in an individual, comprising administering to the individual a population of modified T cells that recognize a cancer-associated antigen, wherein the modified T cells comprise an exogenous nucleic acid molecule encoding an RNA transcript comprising a microRNA (miRNA) that comprises a seed sequence having the nucleotide sequence of SEQ ID NO: 1, and wherein the modified T cells have a lower T cell receptor (TCR) signaling threshold and/or increased TCR sensitivity to the cancer-associated antigen as compared to T cells not comprising the exogenous miRNA.

### Exemplary Embodiments

Embodiment 1. A method of treating cancer in an individual, comprising administering to the individual a population of modified T cells that recognize a cancer-associated antigen, wherein the modified T cells comprise an exogenous nucleic acid molecule encoding an RNA transcript comprising a microRNA (miRNA) that comprises a seed sequence having the nucleotide sequence of SEQ ID NO: 1, and wherein the modified T cells have a lower T cell receptor (TCR) signaling threshold and/or increased TCR sensitivity to the cancer-associated antigen as compared to T cells not comprising the exogenous miRNA.

Embodiment 2. The method of embodiment 1, wherein the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6.

Embodiment 3. The method of embodiment 2, wherein the miRNA targets each of the mRNAs encoding PTPN11, PTPN22, DUSP5, and DUSP6.

Embodiment 4. The method of any one of embodiments 1-3, wherein the RNA transcript comprises a sequence corresponding to a precursor miRNA (pre-miRNA).

Embodiment 5. The method of any one of embodiments 1-4, wherein the RNA transcript comprises a sequence corresponding to a primary miRNA (pri-miRNA).

Embodiment 6. The method of any one of embodiments 1-5, wherein the RNA transcript comprises a loop region having the nucleotide sequence of SEQ ID NO: 4 or 5, or a variant thereof comprising up to 3 nucleotide substitutions.

Embodiment 7. The method of embodiment 6, wherein the RNA transcript comprises a loop region having the nucleotide sequence of SEQ ID NO: 4 or 5.

Embodiment 8. The method of any one of embodiments 1-7, wherein the RNA transcript comprises a stem region having the nucleotide sequence of SEQ ID NO: 3, or a variant thereof comprising up to 3 nucleotide substitutions.

Embodiment 9. The method of embodiment 8, wherein the RNA transcript comprises a stem region having the nucleotide sequence of SEQ ID NO: 3.

Embodiment 10. The method of embodiment 4, wherein the sequence corresponding to the pre-miRNA has the nucleotide sequence of SEQ ID NO: 6 or 7.

Embodiment 11. The method of embodiment 5, wherein the sequence corresponding to the pri-miRNA has the nucleotide sequence of SEQ ID NO: 8 or 9.

Embodiment 12. The method of any one of embodiments 1-11, further comprising introducing the exogenous nucleic acid molecule into a population of input T cells, thereby generating the population of modified T cells.

Embodiment 13. The method of embodiment 12, wherein the exogenous nucleic acid molecule is introduced by viral transduction, transposition, electroporation, or chemical transfection.

Embodiment 14. A method of treating cancer in an individual, comprising administering to the individual a population of modified T cells that recognize a cancer-associated antigen in the individual, wherein the modified T cells comprise a modification that increases the expression of endogenous miR-181a, and wherein the modified T cells have a lower T cell receptor (TCR) signaling threshold and/or increased TCR sensitivity to the cancer-associated antigen as compared to T cells not modified to increase the expression of endogenous miR-181a.

Embodiment 15. The method of embodiment 14, wherein the modification comprises introducing a nucleic acid molecule encoding a) a fusion protein comprising a nuclease-deficient sequence-guided-endonuclease (SGEN) fused to an activator of RNA polymerase H; and b) a guide nucleotide directing the fusion protein to the promoter region of the miR-181a gene.

Embodiment 16. The method of embodiment 14, wherein the modification comprises inserting a nucleic acid sequence that upregulates miR-181a into the genome of the T cells.

Embodiment 17. The method of embodiment 16, wherein the nucleic acid sequence encodes miR-181a.

Embodiment 18. The method of embodiment 16, wherein the nucleic acid sequence comprises a promoter sequence and is inserted such that the promoter sequence is operably linked to the miR-181a gene.

Embodiment 19. The method of any one of embodiments 16-18, wherein the nucleic acid sequence is inserted by homologous recombination.

Embodiment 20. The method of embodiment 19, wherein the nucleic acid sequence is inserted using CRISPR.

Embodiment 21. The method of embodiment 16 or 17, wherein the nucleic acid sequence is inserted by random integration.

Embodiment 22. The method of embodiment 21, wherein the nucleic acid sequence is inserted by viral transduction.

Embodiment 23. The method of embodiment 14, wherein the modification comprises a modification to the miR-181a gene that leads to an increase in the stability of the mRNA transcript of the miR-181a gene.

Embodiment 24. The method of any one of embodiments 14-23, further comprising modifying a population of input T cells, thereby generating the population of modified T cells.

Embodiment 25. The method of any one of embodiments 1-24, further comprising administering a second therapy or therapeutic agent.

Embodiment 26. The method of embodiment 25, wherein the method further comprises administering a conditioning chemotherapy prior to administration of the modified T cells.

Embodiment 27. The method of embodiment 25, wherein the method further comprises administering a chemotherapeutic agent.

Embodiment 28. The method of embodiment 25, wherein the method further comprises administering an immunotherapeutic agent.

Embodiment 29. The method of embodiment 28, wherein the immunotherapeutic agent is selected from the group consisting of IL-2, IL-7, IL-15, IL-12 and IL-21.

Embodiment 30. The method of any one of embodiments 1-29, wherein the modified T cells are autologous to the individual.

Embodiment 31. The method of embodiment 30, further comprising isolating T cells from the individual, thereby generating the autologous input T cells.

Embodiment 32. The method of embodiment 31, wherein the T cells are isolated from a solid tumor in the individual.

Embodiment 33. The method of any one of embodiments 1-29, wherein the modified T cells are allogeneic to the individual.

Embodiment 34. The method of any one of embodiments 1-33, wherein the dose of the modified T cells administered to the individual is at least about 1×10⁵ cells per kilogram body weight of the individual.

Embodiment 35. The method of any one of embodiments 1-33, wherein the dose of the modified T cells administered to the individual is at least about 1×10⁷ cells.

Embodiment 36. The method of any one of embodiments 1-33, wherein the dose of the modified T cells administered to the individual is at least about 1×10⁷ cells/m² body surface area of the individual.

Embodiment 37. The method of any one of embodiments 1-36, wherein the modified T cells are administered to the individual by intravenous, intraperitoneal, or subcutaneous injection.

Embodiment 38. The method of any one of embodiments 1-37, wherein the individual is human.

Embodiment 39. The method of any one of embodiments 1-38, wherein the cancer is a solid tumor.

Embodiment 40. The method of embodiment 39, wherein the cancer is pancreatic cancer, breast cancer, or melanoma.

Embodiment 41. The method of any one of embodiments 1-40, wherein the cancer is metastatic cancer.

Embodiment 42. A method of producing a population of modified T cells, comprising introducing into a population of input T cells recognizing a cancer-associated antigen an exogenous nucleic acid molecule encoding an miRNA that comprises a seed sequence having the nucleotide sequence of SEQ ID NO: 1, wherein the modified T cells have a lower T cell receptor (TCR) signaling threshold and/or increased TCR sensitivity to the cancer-associated antigen.

Embodiment 43. The method of embodiment 42, wherein the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6.

Embodiment 44. The method of embodiment 43, wherein the miRNA targets each of the mRNAs encoding PTPN11, PTPN22, DUSP5, and DUSP6.

Embodiment 45. The method of any one of embodiments 42-44, wherein the RNA transcript comprises a sequence corresponding to a precursor miRNA (pre-miRNA).

Embodiment 46. The method of any one of embodiments 42-45, wherein the RNA transcript comprises a sequence corresponding to a primary miRNA (pri-miRNA).

Embodiment 47. The method of any one of embodiments 42-46, wherein the RNA transcript comprises a loop region having the nucleotide sequence of SEQ ID NO: 4 or 5, or a variant thereof comprising up to 3 nucleotide substitutions.

Embodiment 48. The method of embodiment 47, wherein the RNA transcript comprises a loop region having the nucleotide sequence of SEQ ID NO: 4 or 5.

Embodiment 49. The method of any one of embodiments 42-48, wherein the RNA transcript comprises a stem region having the nucleotide sequence of SEQ ID NO: 3, or a variant thereof comprising up to 3 nucleotide substitutions.

Embodiment 50. The method of embodiment 49, wherein the RNA transcript comprises a stem region having the nucleotide sequence of SEQ ID NO: 3.

Embodiment 51. The method of embodiment 45, wherein the sequence corresponding to the pre-miRNA has the nucleotide sequence of SEQ ID NO: 6 or 7.

Embodiment 52. The method of embodiment 46, wherein the sequence corresponding to the pri-miRNA has the nucleotide sequence of SEQ ID NO: 8 or 9.

Embodiment 53. The method of any one of embodiments 42-52, wherein the exogenous nucleic acid molecule is introduced by viral transduction, transposition, electroporation, or chemical transfection.

Embodiment 54. A method of producing a population of modified T cells, comprising introducing into a population of input T cells recognizing a cancer-associated antigen a modification that increases the expression of endogenous miR-181a, wherein the modified T cells have a lower TCR signaling threshold and/or increased TCR sensitivity to the cancer-associated antigen.

Embodiment 55. The method of embodiment 54, wherein the modification comprises introducing a nucleic acid molecule encoding a) a fusion protein comprising a nuclease-deficient sequence-guided endonuclease (SGEN) fused to an activator of RNA polymerase II; and b) a guide nucleotide directing the fusion protein to the promoter region of the miR-181a gene.

Embodiment 56. The method of embodiment 54, wherein the modification comprises inserting a nucleic acid sequence that upregulates miR-181a into the genome of the T cells.

Embodiment 57. The method of embodiment 56, wherein the nucleic acid sequence encodes miR-181a.

Embodiment 58. The method of embodiment 56, wherein the nucleic acid sequence comprises a promoter sequence and is inserted such that the promoter sequence is operably linked to the miR-181a gene.

Embodiment 59. The method of any one of embodiments 56-58, wherein the nucleic acid sequence is inserted by homologous recombination.

Embodiment 60. The method of embodiment 59, wherein the nucleic acid sequence is inserted using CRISPR.

Embodiment 61. The method of embodiment 56 or 57, wherein the nucleic acid sequence is inserted by random integration.

Embodiment 62. The method of embodiment 61, wherein the nucleic acid sequence is inserted by viral transduction.

Embodiment 63. The method of embodiment 54, wherein the modification comprises a modification to the miR-181a gene that leads to an increase in the stability of the mRNA transcript of the miR-181a gene.

Embodiment 64. The method of any one of embodiments 42-63, wherein the input T cells are isolated from a solid tumor in an individual.

Embodiment 65. The method of embodiment 64, further comprising isolating T cells from the solid tumor, thereby generating the population of input T cells.

Embodiment 66. A population of modified T cells prepared by the method of any one of embodiments 42-65.

Embodiment 67. A composition comprising the population of modified T cells of embodiment 66.

Embodiment 68. A pharmaceutical composition comprising the population of modified T cells of embodiment 66 and a pharmaceutically acceptable carrier.

Embodiment 69. A polyclonal population of modified T cells that recognize two or more cancer-associated antigens in an individual, wherein the modified T cells comprise an exogenous nucleic acid molecule encoding a microRNA (miRNA) that comprises a seed sequence having the nucleotide sequence of SEQ ID NO: 1, and wherein the modified T cells have a lower T cell receptor (TCR) signaling threshold and/or increased TCR sensitivity to the cancer-associated antigen.

Embodiment 70. A polyclonal population of modified T cells that recognize two or more cancer-associated antigens in an individual, wherein the modified T cells comprise a modification that increases the expression of endogenous miR-181a, and wherein the modified T cells have a lower T cell receptor (TCR) signaling threshold and/or increased TCR sensitivity to the cancer-associated antigen.

Embodiment 71. A pharmaceutical composition comprising the polyclonal population of modified T cells of embodiment 69 or 70 and a pharmaceutically acceptable carrier.

Embodiment 72. A commercial batch of the polyclonal population of modified T cells of embodiment 69 or 70.

Embodiment 73. A needle filled with the polyclonal population of modified T cells of embodiment 69 or 70.

### EXAMPLES

The following examples are included for illustrative purposes only and are not intended to limit the scope of the invention.

### Example 1A. Potentiation of TIL Cytotoxicity Against Pancreatic Cancer Cells Mediated by Mir-181

In order to characterize the effect of miR-181 expression in tumor infiltrating lymphocytes (TILs) on their cytotoxicity against pancreatic cancer cells, TILs were harvested from mice bearing orthotopically implanted KPC tumor cells, and either mock-transduced (control TILs) or transduced with a virus for the overexpression of miR-181 (miR-181 TILs). KPC tumor cells were derived from a genetically engineered mouse model (KrasLSL.G12D/+; p53R172H/+; PdxCretg/+) of pancreatic ductal adenocarcinoma (PDA)

### In Vitro Characterization

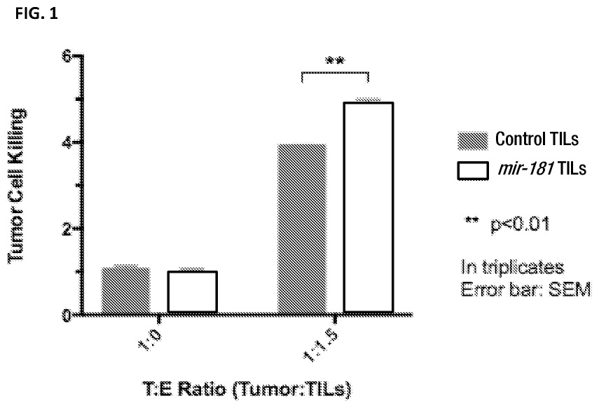

Control TILs and miR-181 TILs were incubated with KPC cells (with Luciferase reporter) at target-to-effector ratios of 1:0 and 1:1.5. Specific lysis was determined after 16 hr incubation by measuring luciferase activity in culture media. As shown in FIG. 1, TILs transduced to overexpress miR-181 showed greater tumor cell killing than the control TILs (in triplicate; t-test, p<0.01).

### In Vivo Characterization

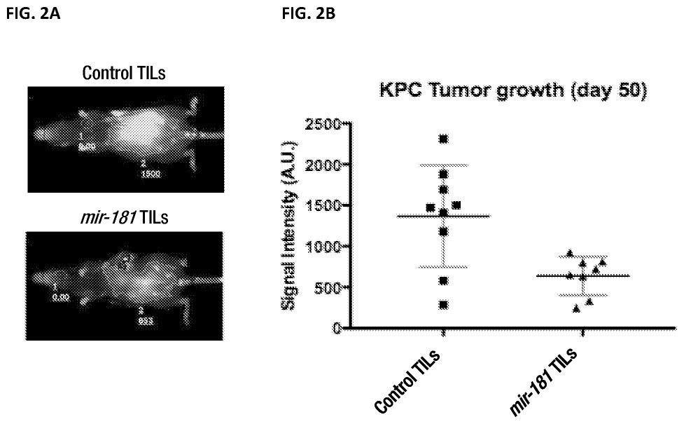

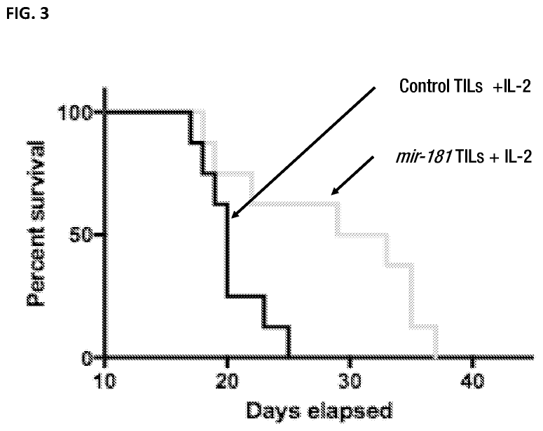

The in vivo antitumor activity of control TILs and miR-181 TILs was evaluated in mice bearing orthotopic KPC tumors. At one-week post-tumor implantation, mice were randomized to two groups receiving: (i) control TILs+IL-2 or (ii) miR-181 TILs+IL-2. The animals were treated immediately after randomization by injecting 1×10⁶ control or miR-181 TILs per mouse intravenously (i.v.) once per week, for three doses. The mice were closely monitored for general health condition, possible adverse response, if any, and changes in tumor volume. Both control and miR-181 TILs were well-tolerated at the current dose and schedule. The mice were injected with IRdye 800CW-2-deoxy-Glucose (1 μg/mouse) at day 21 after tumor implantation and imaged at 48 hour after 2-DG dye injection using a Licor Pearl Trilogy small animal imaging system and signal intensity was quantified as an indication of KPC tumor growth (FIGS. 2A and 2B). Mice injected with the miR-181 TILs showed less tumor growth than mice injected with control TILs. The survival curves for the mock chemotherapy conditioning groups and the chemotherapy conditioning groups are shown in FIG. 3. Treatment with the miR-181 TILs extended survival of mice bearing pancreatic tumors to a greater extent than the control TILs without chemotherapy conditioning.

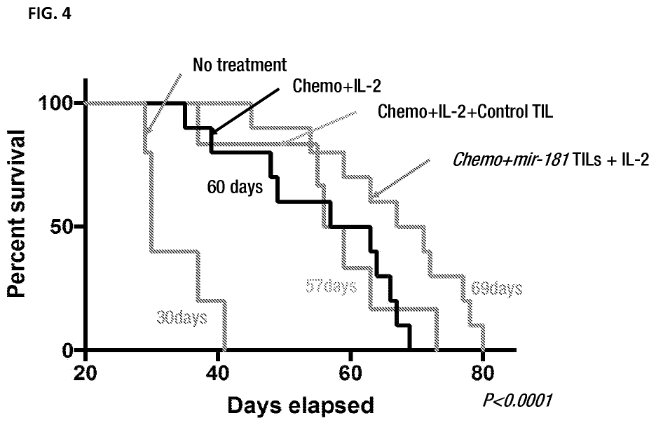

In another study, the antitumor activity of combined chemotherapy conditioning and TIL treatment was evaluated in mice bearing orthotopic KPC tumors. At one-week post-tumor implantation, animals were randomized to four groups receiving: (i) no treatment, (ii) chemo+IL-2, (iii) chemo+control TILs+IL-2, or (iv) chemo+miR-181 TILs+IL-2. 1×10⁶ control or miR-181 TILs per mouse were injected intravenously once every two weeks, for three doses. The survival curves for the treatment groups are shown in FIG. 4. The miR-181 TILs extended survival of mice bearing pancreatic tumors to a greater extent than the control TILs with chemotherapy conditioning, and the antitumor activity of the miR-181 TILs was further enhanced with chemotherapy conditioning.

### Example 1B. Potentiation of TIL Cytotoxicity Against Melanoma Cancer Cells Mediated by miR-181

In order to characterize the effect of miR-181 expression on the CTL activity of tumor antigen-specific T cells, Pmel T cells were harvested from the spleen of Pmel transgenic mice. This transgenic strain carries a rearranged T cell receptor transgene specific for the mouse homologue (pmel^{Si} or pmel-17) of human premelanosome protein (referred to as PMEL, SILV or gp100), and the T lymphocyte specific Thy1^{a} (Thy1.1) allele. We examined the CTL activity of control or miR-181 transduced Pmel T cells against B16F0 melanoma tumor cells.

### In Vitro Characterization

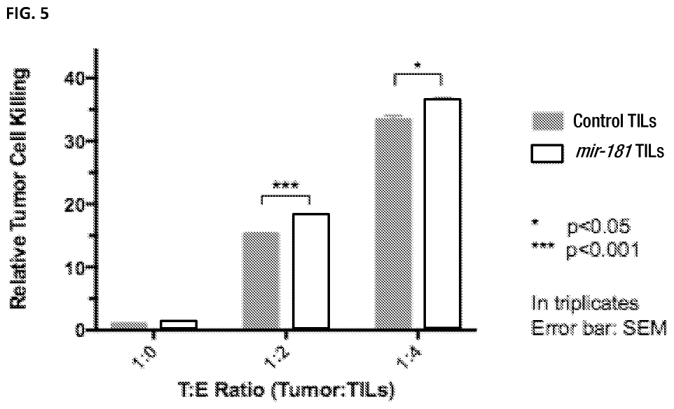

Control Pmel T cells and miR-181 Pmel T cells were incubated with B16F0 cells (with Luciferase reporter) at target-to-effector ratios of 1:0, 1:2, and 1:4. Specific lysis was measured after 16 hr incubation by measuring the luciferase activity in culture media. As shown in FIG. 5, TILs transduced to overexpress miR-181 showed greater tumor cell killing than the control TILs at target-to-effector ratios of 1:2 (in triplicate; t-test, p<0.001) and 1:4 (in triplicate; t-test, p<0.05).

### In Vivo Characterization

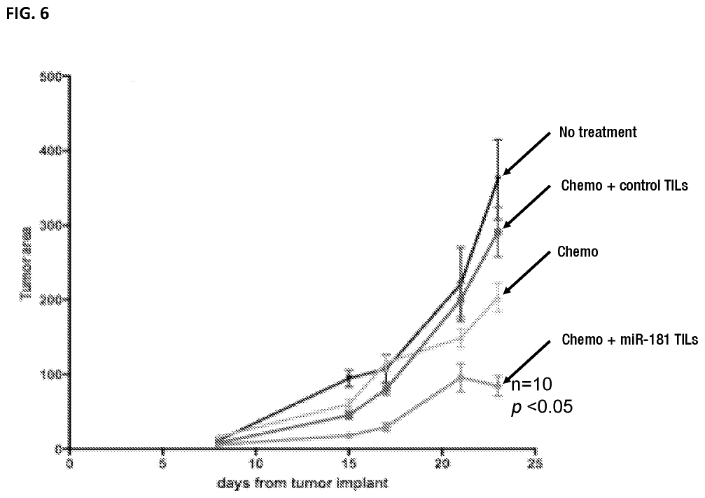

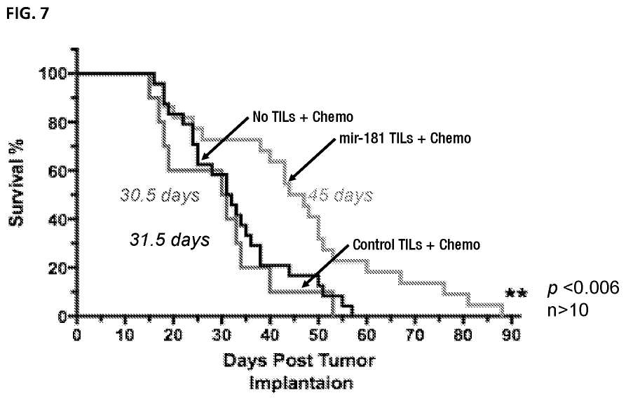

The in vivo antitumor activity of control Pmel T cells and miR-181 Pmel T cells was evaluated in mice bearing B16F0 melanoma tumors. On day 8 post-tumor implantation, animals were randomized to four groups receiving: (i) no treatment, (ii) chemotherapy conditioning, (iii) chemotherapy conditioning+control TILs, or (iv) chemotherapy conditioning+miR-181 TILs. Mice did not receive IL-2 injection. The animals were treated one day after chemotherapy conditioning and injected with 1×10⁶ control or miR-181 TILs per mouse intravenously (i.v.), or mock injection, once per week, for three doses. The mice were closely monitored for general health condition, possible adverse response, if any, and changes in tumor volume. Both control and miR-181 TILs were well-tolerated at the current dose and schedule. As shown in FIG. 6, mice injected with the miR-181 TILs showed less tumor growth than any of the other groups, including group (iii) with control TIL injection. The survival curves for groups (ii), (iii), and (iv) are shown in FIG. 7. Mice injected with the miR-181 TILs showed greater survival than any of the other groups, including group (iii) with control TIL injection (n>10; t-test, p<0.006).

### Example 1C. Potentiation of TIL Cytotoxicity Against Pancreatic Cancer Cells Mediated by miR-181

In order to characterize the effect of miR-181 expression in tumor infiltrating lymphocytes (TILs) on their cytotoxicity against breast cancer cells, TILs were harvested from mice bearing orthotopic 4T1 mammary tumor cells, and either mock-transduced (control TILs) or transduced with a virus for the overexpression of miR-181 (miR-181 TILs).

### In Vitro Characterization

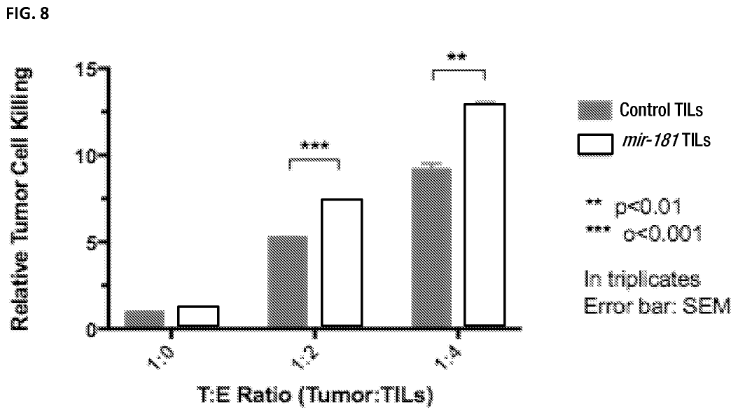

Control TILs and miR-181 TILs were incubated with 4T1 cells (with Luciferase reporter) at target-to-effector ratios of 1:0, 1:2, and 1:4. Specific lysis was measured after 16 hr incubation by measuring the luciferase activity in culture media. As shown in FIG. 8, TILs transduced to overexpress miR-181 showed greater tumor cell killing than the control TILs at target-to-effector ratios of 1:2 (in triplicate; t-test, p<0.001) and 1:4 (in triplicate; t-test, p<0.01).

### In Vivo Characterization

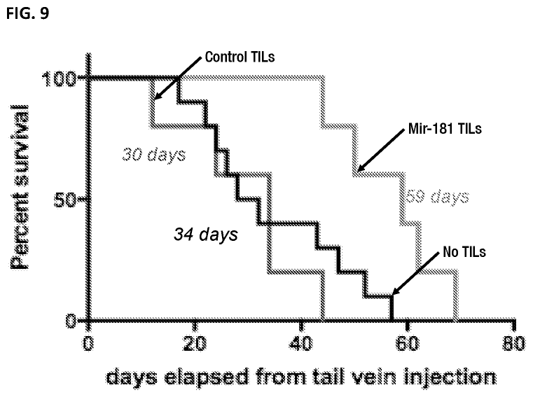

The in vivo control of tumor metastasis with control TILs and miR-181 TILs was evaluated in mice bearing orthotopic 4T1 tumors. On day 8 post-tumor implantation, animals were randomized to three groups receiving: (i) no treatment, (ii) control TILs, or (iii) miR-181 TILs. No IL-2 and no chemotherapy conditioning was utilized. The animals were treated once immediately after randomization by injecting 1×10⁶ control or miR-181 TILs per mouse intravenously (i.v.), or mock injection. The mice were closely monitored for general health condition, possible adverse response, if any, and changes in tumor volume. Both control and miR-181 TILs were well-tolerated at the current dose and schedule. The survival curves for groups (i), (ii), and (iii) are shown in FIG. 9. Mice injected with the miR-181 TILs showed greater survival than either of the other groups.

### Example 2. Characterization of TILs Overexpressing miR-18

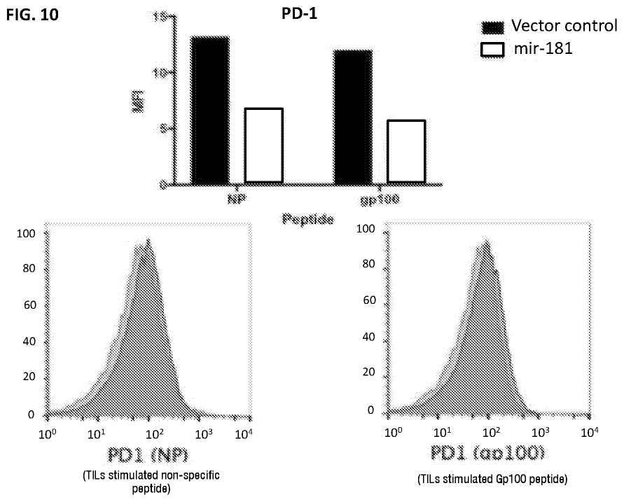

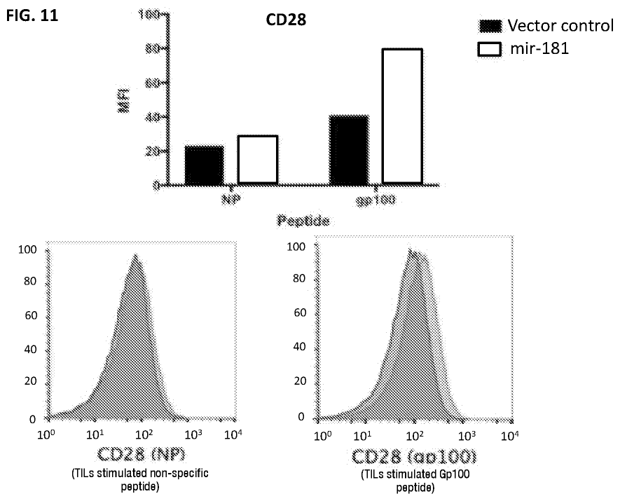

In order to further characterize the effect of miR-181 expression in tumor infiltrating lymphocytes (TILs), the surface expression levels of two regulators of T cell activation, PD-1 and CD28, were evaluated. Pmel T cells transduced with either a control virus (409) or miR-181 virus were activated by antigen presenting cells loaded with either a null antigen or gp100 peptide. The cells were analyzed by flow cytometry for either PD-1 expression (FIG. 10) or CD28 expression (FIG. 11). As shown in FIG. 10, Pmel T cells overexpressing miR-181 had much less PD-1 expression than control Pmel T cells regardless of antigen activation. As shown in FIG. 11, a greater increase of CD28 expression was observed in miR-181 Pmel T cells activated with GP100 peptide antigen but not in other control Pmel T cells. Taken together, these results suggest that TILs overexpressing miR-181 could be more responsive and less prone to suppression by checkpoint inhibition than TILs expressing typical levels of miR-181, and thus may be more effective for adoptive cell therapy.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ | | |
|---|---|---|
| ID | | |
| NO | SEQUENCE | ANNOTATION |
| 1 | ACAUUCA | miR-181a-1 |
| | | seed |
| 2 | AACAUUCAACGCUGUCGGUGAGU | miR-181a-1 |
| 3 | ACCATCGACCGTTGATTGTA | miR-181a-1 |
| | | stem |
| 4 | TTGGAATTCAAATAAAA | miR-181a-1 |
| | | loop |
| 5 | TTGGGATTTGAAAAA | miR-181a-2 |
| | | loop |
| 6 | AACATTCAACGCTGTCGGTGAGTTTGGAATTCAA | miR-181a-1 |
| | ATAAAAACCATCGACCGTTGATTGTA | pre-miRNA |
| 7 | AGAAGGGCTATCAGGCCAGCCTTCAGAGGACTCC | miR-181a-2 |
| | AAGGAACATTCAACGCTGTCGGTGAGTTTGGGAT | pre-miRNA |
| | TTGAAAAAACCACTGACCGTTGACTGTACCTTGG | |
| | GGFCCTTA | |
| 8 | CTCGAGTGTGACAGGTTTGGTTAAAGGATTGGGC | miR-181a-1 |
| | TTTCCTCTGCCTCCCTCCTGCTCCAGACTCCCAC | pri-miRNA |
| | AGATACTGTTTAAATCAGCACATCTCTGCCTCAC | |
| | AGGTTGCTTCAGTGAACATTCAACGCTGTCGGTG | |
| | AGTTTGGAATTCAAATAAAAACCATCGACCGTTG | |
| | ATTGTACCCTATAGCTAACCATCATCTACTCCAT | |
| | GGCCCTCTGCGTTTGCTGAAGACAGAACCGCAAA | |
| | GCAGGACCCGACAGGATTCTTTTTTAATTAAGAA | |
| | TTCCTAGGAATTCTTGCCAAACCTACAGGTGGGG | |
| | TCTTTCATTCCCCCCTTTTTCTGGAGACTAAATA | |
| | AAATCTTTTATTTTATCGATAAGCTTGGCTGCAG | |
| | GTCGACGCGGCCGC | |
| 9 | TTTAAATACTCTCGACTTGAAACCCAGAGAGGAA | miR-181a-2 |
| | TGTAAGAGCATCCATCAGCGGTGGTCTCACTGCT | pri-miRNA |
| | CACTGGTTCTTGGGATGTGGATGGGAGAATGAAG | |
| | AAGGGCTATCAGGCCAGCCTTCAGAGGACTCCAA | |
| | GGAACATTCAACGCTGTCGGTGAGTTTGGGATTT | |
| | GAAAAAACCACTGACCGTTGACTGTACCTTGGGG | |
| | TCCTTACAGACGACACTACATTTCCTGAAGCAAA | |
| | AGAGCAAGCTGTACCTTCACATGTCACATGAGTT | |
| | CACCAGAAATGGTCCTGCAATCCCCCAAATGTGG | |
| | TCCA | |

1: A method of treating cancer in an individual, comprising administering to the individual a population of modified T cells that recognize a cancer-associated antigen, wherein the modified T cells comprise an exogenous nucleic acid molecule encoding an RNA transcript comprising a microRNA (miRNA) that comprises a seed sequence having the nucleotide sequence of SEQ ID NO: 1, and wherein the modified T cells have a lower T cell receptor (TCR) signaling threshold and/or increased TCR sensitivity to the cancer-associated antigen as compared to T cells not comprising the exogenous miRNA.

2: The method of claim 1, wherein the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6.

3: The method of claim 2, wherein the miRNA targets each of the mRNAs encoding PTPN11, PTPN22, DUSP5, and DUSP6.

4: The method of claim 1, wherein the RNA transcript comprises a sequence corresponding to a precursor miRNA (pre-miRNA).

5: The method of claim 1, wherein the RNA transcript comprises a sequence corresponding to a primary miRNA (pri-miRNA).

6: The method of claim 1, wherein the RNA transcript comprises a loop region having the nucleotide sequence of SEQ ID NO: 4 or 5, or a variant thereof comprising up to 3 nucleotide substitutions.

7: The method of claim 1, wherein the RNA transcript comprises a stem region having the nucleotide sequence of SEQ ID NO: 3, or a variant thereof comprising up to 3 nucleotide substitutions.

8: The method of claim 4, wherein the sequence corresponding to the pre-miRNA has the nucleotide sequence of SEQ ID NO: 6 or 7.

9: The method of claim 5, wherein the sequence corresponding to the pri-miRNA has the nucleotide sequence of SEQ ID NO: 8 or 9.

10: The method of claim 1, further comprising introducing the exogenous nucleic acid molecule into a population of input T cells, thereby generating the population of modified T cells.

11: A method of treating cancer in an individual, comprising administering to the individual a population of modified T cells that recognize a cancer-associated antigen in the individual, wherein the modified T cells comprise a modification that increases the expression of endogenous miR-181a, and wherein the modified T cells have a lower T cell receptor (TCR) signaling threshold and/or increased TCR sensitivity to the cancer-associated antigen as compared to T cells not modified to increase the expression of endogenous miR-181a.

12: The method of claim 11, wherein the modification comprises introducing a nucleic acid molecule encoding a) a fusion protein comprising a nuclease-deficient sequence-guided endonuclease (SGEN) fused to an activator of RNA polymerase II; and b) a guide nucleotide directing the fusion protein to the promoter region of the miR-181a gene.

13: The method of claim 11, wherein the modification comprises inserting a nucleic acid sequence that upregulates miR-181a into the genome of the T cells.

14: The method of claim 13, wherein the nucleic acid sequence encodes miR-181a.

15: The method of claim 13, wherein the nucleic acid sequence comprises a promoter sequence and is inserted such that the promoter sequence is operably linked to the miR-181a gene.

16: The method of claim 11, further comprising modifying a population of input T cells, thereby generating the population of modified T cells.

17: The method of claim 1, further comprising administering a second therapy or therapeutic agent.

18: The method of claim 17, wherein the method further comprises administering a) a conditioning chemotherapy prior to administration of the modified T cells, b) a chemotherapeutic agent, or c) an immunotherapeutic agent.

19: The method of claim 1, wherein the modified T cells are autologous to the individual.

20: The method of claim 19, wherein the T cells are isolated from a solid tumor in the individual.

21: The method of claim 1, wherein the modified T cells are allogeneic to the individual.

22: The method of claim 1, wherein the individual is human.

23: The method of claim 1, wherein the cancer is a solid tumor.

24: A method of producing a population of modified T cells, comprising introducing into a population of input T cells recognizing a cancer-associated antigen an exogenous nucleic acid molecule encoding an miRNA that comprises a seed sequence having the nucleotide sequence of SEQ ID NO: 1, wherein the modified T cells have a lower T cell receptor (TCR) signaling threshold and/or increased TCR sensitivity to the cancer-associated antigen.

25: The method of claim 24, wherein the miRNA targets a plurality of T cell mRNAs selected from the group consisting of mRNAs encoding tyrosine-protein phosphatase non-receptor type (PTPN) 11 (PTPN11), PTPN22, dual specificity protein phosphatase (DUSP) 5 (DUSP5), and DUSP6.

26: The method of claim 25, wherein the miRNA targets each of the mRNAs encoding PTPN11, PTPN22, DUSP5, and DUSP6.

27: The method of claim 24, wherein the RNA transcript comprises a sequence corresponding to a precursor miRNA (pre-miRNA).

28: The method of claim 24, wherein the RNA transcript comprises a sequence corresponding to a primary miRNA (pri-miRNA).

29: The method of claim 24, wherein the RNA transcript comprises a loop region having the nucleotide sequence of SEQ ID NO: 4 or 5, or a variant thereof comprising up to 3 nucleotide substitutions.

30: The method of claim 24, wherein the RNA transcript comprises a stem region having the nucleotide sequence of SEQ ID NO: 3, or a variant thereof comprising up to 3 nucleotide substitutions.

31: The method of claim 27, wherein the sequence corresponding to the pre-miRNA has the nucleotide sequence of SEQ ID NO: 6 or 7.

32: The method of claim 28, wherein the sequence corresponding to the pri-miRNA has the nucleotide sequence of SEQ ID NO: 8 or 9.

33: A method of producing a population of modified T cells, comprising introducing into a population of input T cells recognizing a cancer-associated antigen a modification that increases the expression of endogenous miR-181a, wherein the modified T cells have a lower TCR signaling threshold and/or increased TCR sensitivity to the cancer-associated antigen.

34: A composition comprising a population of modified T cells prepared by the method of claim 24.

35: A polyclonal population of modified T cells that recognize two or more cancer-associated antigens in an individual, wherein the modified T cells comprise an exogenous nucleic acid molecule encoding a microRNA (miRNA) that comprises a seed sequence having the nucleotide sequence of SEQ ID NO: 1, and wherein the modified T cells have a lower T cell receptor (TCR) signaling threshold and/or increased TCR sensitivity to the cancer-associated antigen.

36: A polyclonal population of modified T cells that recognize two or more cancer-associated antigens in an individual, wherein the modified T cells comprise a modification that increases the expression of endogenous miR-181a, and wherein the modified T cells have a lower T cell receptor (TCR) signaling threshold and/or increased TCR sensitivity to the cancer-associated antigen.

37: A pharmaceutical composition comprising the polyclonal population of modified T cells of claim 35 and a pharmaceutically acceptable carrier.
